(19) United States
(12) Patent Application Publication
(10) Pub. No.: US 2025/0295839 A1
(43) Pub. Date: Sep. 25, 2025

(54) 3D PRINTED MATERIALS AND METHODS

(71) Applicant: ARTBIO, Inc., Cambridge, MA (US)

(72) Inventors: Gjermund Fjeld Olsen, Fjerdingby (NO); Tanja Aarvak, Oslo (NO); Judit Tjelmeland Oestby, Kraakstad (NO); Olga Salina, Ski (NO); Stewart Maddison Fox, Cambridge (GB); Tara Clancy, Cambridge (GB); Peter Maurice Wilson, Cambridge (GB)

(73) Assignee: ARTBIO, Inc., Cambridge, MA (US)

(21) Appl. No.: 19/079,716

(22) Filed: Mar. 14, 2025

Related U.S. Application Data
(60) Provisional application No. 63/567,359, filed on Mar. 19, 2024.

(51) Int. Cl. A61L 27/54; A61L 27/56; B33Y 80/00

(52) U.S. Cl. CPC A61L 27/54; A61L 27/56; B33Y 80/00; A61L 2300/102; A61L 2300/44

(57) ABSTRACT

Aspects of the present disclosure generally relate to 3D printed scaffolds for attaching a radionuclide and methods of use thereof. In some embodiments, 3D printed scaffolds comprising a first layer and/or a second layer, and a radionuclide, are provided. In some embodiments, methods for capturing a radionuclide using said 3D printed scaffolds, are provided. In some embodiments, methods of using 3D printed scaffolds with a radionuclide to capture a daughter radionuclide product at a location different than the 3D printed scaffold, are provided.

### CROSS REFERENCE TO RELATED APPLICATIONS

This application claims priority under 35 U.S.C. § 119(e) to U.S. Provisional Application No. 63/567,359, filed Mar. 19, 2024, which is incorporated herein by reference in its entirety for all purposes.

### BACKGROUND

Targeted radionuclide therapy (TRT) has emerged as a leading strategy for targeted delivery of radiation. TRT comprises the use of a radionuclide conjugated to a targeting ligand (“radioligand”) that preferentially binds to a target on a cancer cell (e.g., on the surface of a cancer cell). Once bound to the cancer cell, the radionuclide undergoes radioactive decay, releasing high energy particles (e.g., alpha particles, beta particles, or auger electrons), that destroy the cancer cell to which it is bound. This localizes the radiation to the target cancer cells and reduces known side effects and co-morbidities commonly associated with radiation treatment.

Radionuclides commonly used for TRT, and other medical applications, include actinium-225 (²²⁵Ac), lead-212 (²¹²Pb) and bismuth-212 (²¹²Bi), among others. Production of these radionuclides is typically achieved using radionuclide generators. For example, column-based generators use columns loaded with solid-phase substrates (e.g., cation exchange resin) that adsorb (e.g., capture) a parent radionuclide (e.g., a radionuclide containing the desired target radionuclide in its decay chain), but not the target radionuclide. Decay of the parent radionuclide produces the desired target radionuclide (e.g., parent ²²⁸Th→daughter ²¹²Pb), which can be recovered (e.g., via washing) from the column, separate from the parent radionuclide.

However, current production methods suffer from several drawbacks that reduce the yield and purity of the target radionuclide. For example, radiolytic degradation of generator columns due to high reactivity of parent radionuclides (e.g., ²²⁸Th) results in high energy contaminates (e.g., ²²⁸Th, ²²⁴Ra, etc.) within the target radionuclide (e.g., ²¹²Pb). Further, gaseous radon is prone to diffuse deep within solid-state substrates (e.g., cationic exchange resins), thus reducing the overall yield for any target radionuclide where radon is an intermediate. Thus, improvements are needed.

### SUMMARY

The subject matter of the present disclosure involves, in some cases, interrelated products, alternative solutions to a particular problem, and/or a plurality of different uses of one or more systems and/or articles.

Aspects of the present disclosure relate to a 3D printed scaffold for attaching a radionuclide. In some embodiment, the 3D printed scaffold comprises a first layer comprising a plurality of substantially parallel first structures. The plurality of first structures is, in some embodiments, substantially aligned along a first axis. In some cases, an average spacing between adjacent first structures is at least 0.1 mm and less than or equal to 0.5 mm, according to some embodiments. Additional embodiments include a second layer comprising a plurality of substantially parallel second structures. The plurality of second structures is, in some embodiments, substantially aligned along a second axis. In some cases, an average spacing between adjacent second structures is at least 0.1 mm and less than or equal to 0.5 mm. In some embodiments, a radionuclide is attached to one or more exposed surfaces of the first structures and/or one or more exposed surfaces of the second structures. In some cases, 3D printed scaffold comprising a first layer and a second layer comprises an angle formed between the plurality of first structures substantially aligned along the first axis and the plurality of second structures substantially aligned along the second axis. In some embodiments, the angle formed is greater than 0 degrees and less than or equal to 90 degrees. In some cases, the plurality of first structures has an average cross-sectional area of at least 20 mm² and less than or equal to 2000 mm², and an average width of at least 5 mm and less than or equal to 50 mm. Additionally, or alternatively, in some embodiments, the ratio between the average width of the first structures and the average spacing between adjacent first structures is at least 0.1 mm and less than or equal to 0.5 mm. In other embodiments, the 3D printed scaffold comprises an infill density of at least 10% and less than or equal to 90%.

Other aspects of the disclosure relate to methods for capturing a radionuclide using a 3D printed scaffold. For example, in some embodiments, the methods relate to capturing ²²⁸Th using a 3D printed scaffold. The methods comprise, according to some embodiments, incubating the 3D printed scaffold in a solution comprising a radionuclide that comprises ²²⁰Rn in its decay chain. In some cases, attaching the radionuclide onto one or more exposed surfaces of the 3D printed scaffold produces a radionuclide-loaded 3D printed scaffold. The 3D printed scaffold, according to one set of embodiments, comprises (i) a first layer comprising a plurality of substantially parallel first structures, wherein the plurality of first structures are substantially aligned along a first axis, and wherein an average spacing between adjacent first structures is at least 0.1 mm and less than or equal to 0.5 mm and (ii) a second layer comprising a plurality of substantially parallel second structures, wherein the plurality of second structures are substantially aligned along a second axis, and wherein an average spacing between adjacent second structures is at least 0.1 mm and less than or equal to 0.5 mm; and wherein an angle between the first axis and second axis is greater than 0 degrees and less than or equal to 90 degrees. In some embodiments, the plurality of first structures has an average cross-sectional area of at least 20 mm² and less than or equal to 2000 mm², and an average width of at least 5 mm and less than or equal to 50 mm, wherein a ratio between the average width of the first structures and the average spacing between adjacent first structures is at least 0.1 mm and less than or equal to 0.5 mm, and wherein the 3D printed scaffold comprises an infill density of at least 10% and less than or equal to 90%.

Other aspects of the present disclosure relate to methods of using a radionuclide-loaded 3D printed scaffold to collect a gaseous intermediate at a location different than the 3D printed scaffold. In some embodiments, the methods comprise placing a radionuclide-loaded 3D printed scaffold into a radionuclide generator, allowing the radionuclide (e.g., parent radionuclide) to decay to gaseous ²²⁰Rn, allowing the gaseous ²²⁰Rn to diffuse to a location different than the 3D printed scaffold, and collecting the ²²⁰Rn at the location different than the scaffold. In one set of embodiments, the radionuclide-loaded 3D printed scaffold comprises a first layer comprising a plurality of substantially parallel first structures. The plurality of first structures, according to some embodiments, are substantially aligned along a first axis. In some embodiments, the average spacing between adjacent first structures is at least 0.1 mm and less than or equal to 0.5 mm.

In some embodiments, the radionuclide-loaded 3D printed scaffold comprises a second layer comprising a plurality of substantially parallel second structures. The plurality of second structures, according to some embodiments, are substantially aligned along a second axis. In some embodiments, the average spacing between adjacent second structures is at least 0.1 mm and less than or equal to 0.5 mm. Additionally, in some embodiments, the radionuclide loaded onto the 3D printed scaffold comprises ²²⁰Rn in its decay chain.

In another aspect, the present disclosure encompasses methods of making one or more of the embodiments described herein, for example, a radionuclide-loaded 3D printed scaffold. In still another aspect, the present disclosure encompasses methods of using one or more of the embodiments described herein, for example, a radionuclide-loaded 3D printed scaffold.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is an exemplary schematic illustration depicting a cross-sectional view of a 3D printed scaffold comprising a first layer and a second layer, according to some embodiments;

FIG. 1B as an exemplary schematic illustration depicting a cross-sectional view of a 3D printed scaffold comprising a first layer comprising a radionuclide and a second layer comprising a radionuclide, according to some embodiments;

FIG. 2 is an exemplary schematic illustration depicting a top view of a first layer of a 3D printed scaffold, the first layer comprising a plurality of substantially parallel first structures, and a cross-sectional area of a first structure, according to some embodiments;

FIG. 3 is an exemplary schematic illustration depicting a top view of a second layer of a 3D printed scaffold, the second layer comprising a plurality of substantially parallel second structures, and a cross-sectional area of a second structure, according to some embodiments;

FIG. 4A is an exemplary schematic illustration depicting a top view of a 3D printed scaffold comprising a first layer comprising a plurality of substantially parallel first structures aligned along a first axis and a second layer comprising a plurality of substantially parallel second structures aligned along a second axis; FIG. 4A also shows a first angle formed between the first axis and the second axis, according to some embodiments;

FIG. 4B is an exemplary schematic illustration depicting a top view of a 3D printed scaffold comprising a first layer comprising a plurality of substantially parallel first structures aligned along a first axis and a second layer comprising the plurality of substantially parallel second structures aligned along a second axis; FIG. 4B also shows a second angle formed between the first axis and the second axis, according to some embodiments;

FIG. 4C is an exemplary schematic illustration depicting a top view of a 3D printed scaffold comprising a first layer, a second layer, and a radionuclide. FIG. 4D is an exemplary schematic illustration depicting a cross-sectional view of FIG. 4C, according to some embodiments;

FIGS. 5A-5C show the observed pore shape formed in the spacings between the first and second substantially parallel structures in the first and second layers aligned along a first axis and second axis, respectively, when viewed from the top position for 3D printed scaffolds where the angle between the first axis and second axis is between 0 and 90 degrees. The figures also show the rectangle pores (FIG. 5A), square pores (FIG. 5B), and diamond pores (FIG. 5C), according to some embodiments;

FIG. 6A shows an exemplary schematic illustration of a top view of a nonporous top and/or bottom layer of a 3D printed scaffold. FIG. 6B shows a cross-sectional view of FIG. 6A, according to some embodiments;

FIG. 7A-7C show a top view (FIG. 7A), a cross-sectional view along the xz-axis (FIG. 7B) and a cross-sectional view along the xy-axis (FIG. 7C) of an exemplary 3D printed scaffold comprising a nonporous bottom layer, a nonporous side layer, a nonporous top layer, and a first and/or second layer, according to some embodiments;

FIGS. 8A-8B show a top view of an exemplary 3D printed scaffold comprising a nonporous bottom layer, a nonporous side layer and a first and/or second layer;

FIGS. 9A-9C shows a side view of an exemplary radionuclide generator for housing an exemplary 3D printed scaffold, according to some embodiments, and

FIG. 10 shows a top view of exemplary 3D printed scaffolds of increasing infill density (e.g., from the top to the bottom), according to some embodiments.

FIG. 11 depicts a diagram of an exemplary thorium series decay chain beginning with thorium-232 (²³²Th). The half-life of each radionuclide in the decay chain is noted in the figure. Note that the radon-220 radionuclide (²²⁰Rn) rapidly decays (e.g., a half-life of 55.6 seconds) into polonium-216 (²¹⁶Po), which further quickly decays (e.g., a half-life of 0.145 seconds) into a lead-212 radionuclide (²¹²Pb). The approximately 10.6 hour half-life of ²¹²Pb may be desirable for various applications. It will further be noted from FIG. 11 that ²²⁰Rn may be a gas at ambient pressure and temperature. Accordingly, it will be appreciated that when a source including a precursor radionuclide to ²²⁰Rn (e.g., ²²⁴Ra, ²²⁸Th, ²²⁸Ra, ²³²Th, or ²²⁸Ac) is exposed to a portion of a scaffold described herein (e.g., an interior surface, a structure (e.g., first structure, second structure)), the precursor radionuclide may decay into the gaseous ²²⁰Rn. The gaseous ²²⁰Rn may then decay into (e.g., via ²¹⁶Po)²¹²Pb and may deposit onto the exposed portion of the scaffold as ²¹²Pb. It should be appreciated that any of the radionuclides in this decay scheme may be present in one or more embodiments described herein.

### DETAILED DESCRIPTION

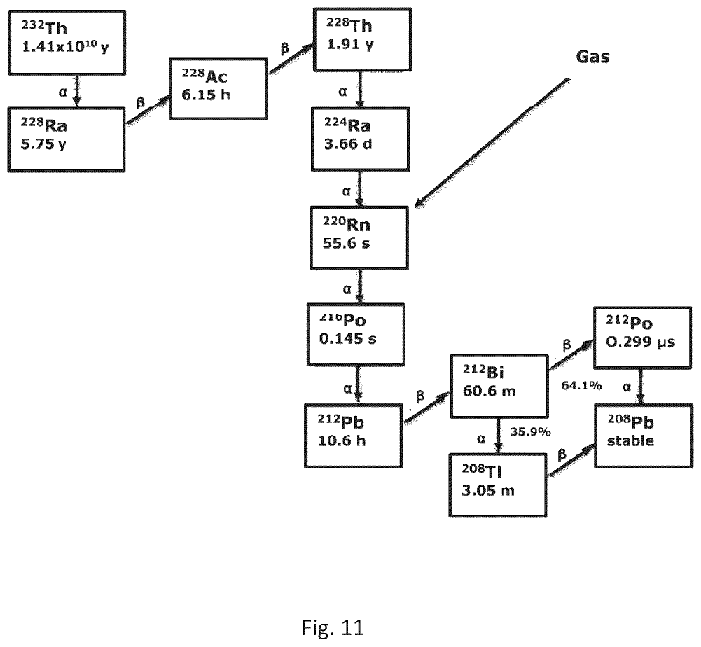

3D printed scaffolds and associated methods for the production of therapeutic radionuclides are generally described. In some embodiments, the 3D printed scaffolds disclosed herein comprise a first layer comprising a plurality of substantially parallel first structures aligned along a first axis. In some embodiments, the 3D printed scaffolds comprise a second layer comprising a plurality of substantially parallel second structures aligned along a second axis. Additional layers are also possible. One or more radionuclides may be attached to one or more exposed surfaces of the first structures and/or second structures. In some embodiments, methods are described for capturing a radionuclide (e.g., a parent radionuclide) using any one of the 3D printed scaffolds disclosed herein. Methods for using the 3D printed scaffolds disclosed herein to capture a daughter radionuclide product at a location different than the 3D printed scaffold are also provided. For example, in some embodiments, the 3D printed scaffold is loaded with a radionuclide (e.g., parent radionuclide), for example, Radium-224 (²²⁴Rn). The ²²⁴Rn will spontaneously decay to produce an alpha particle and daughter radionuclide, e.g., Radon-220 (²²⁰Ra). Daughter radionuclide ²²⁰Ra, which is a gas, may diffuse out of and away from the 3D printed scaffold to a location that is different from the 3D printed scaffold. Once at the new location, the first daughter radionuclide, e.g., ²²⁰Ra, may further decay to produce an alpha particle and a second daughter radionuclide, e.g., Polonium-216 (²¹⁶Po) (daughter radionuclide of ²²⁰Ra and granddaughter radionuclide of ²²⁴Rn). Because ²¹⁶Po is an unstable isotope, with a half-life of only 0.14 seconds, it decays to a third daughter radionuclide, e.g., Lead-212 (²¹²Pb), near instantaneously (daughter of ²¹⁶Po, granddaughter of ²²⁰Ra, and great granddaughter of ²²⁴Rn) (FIG. 11).

Certain existing methods for producing target radionuclides from a parent radionuclide suffer from several drawbacks that collectively reduce the overall yield and purity of the desired therapeutic radionuclide (e.g., ²¹²Pb). For example, radiolytic degradation of the solid-state substrate (e.g., due to high energy of the adsorbed parent radionuclide) can result in high energy contaminants in the final product (e.g., a mixture of therapeutic ²¹²Pb and contaminant ²²⁸Th). Such contaminants may lead to significant comorbidities, and even death, in patients receiving TRT.

Accordingly, one aspect of the present disclosure is directed to the discovery that the use of the 3D printed scaffolds disclosed herein, reduces and/or eliminates radiolytic degradation of the solid support, and hence, reduces and/or eliminates contamination of high energy radionuclides within the final product. For example, in some embodiments, the 3D printed scaffolds disclosed herein are comprised of zirconia and/or quartz (SiO₂), both of which are chemically inert and unlikely to undergo radiolytic decay from the adsorbed parent radionuclide.

Additionally, the 3D printed scaffolds disclosed herein comprise relatively high surface areas for adsorption of radionuclides (e.g., parent radionuclide) and relatively high porosities allowing transport of gas-based intermediates (e.g., ²²⁰Rn), which emanate from the radionuclide (e.g., parent radionuclide) (e.g., ²²⁴Ra), to readily diffuse through the porous scaffold to a location different from the scaffold before decaying into other radionuclides. It has been discovered that collectively, these features eliminate or reduce many of the problems associated with traditional radionuclide generators, thereby facilitating relatively high yield production of substantially pure daughter radionuclides (e.g., ²¹²Pb). For instance, the materials and methods described herein can eliminate the need to perform chemical extractions to separate the daughter from parent radionuclides and/or may have high resistance to radiolytic degradation which may reduce the formation of impurities. The materials and methods described herein may be particularly useful when used in combination with a radionuclide generator, e.g., configured to collect a gaseous intermediate radionuclide at a location different than the location of the parent radionuclide, as described in more detail in U.S. Provisional Patent Application No. 63/458,296, entitled “Radionuclide Generator” filed on Apr. 10, 2023, and U.S. patent application Ser. No. 17/756,802, entitled “Production of Highly Purified 121Pb” filed on Jun. 2, 2022, both of which are hereby incorporated by reference in their entireties for all purposes.

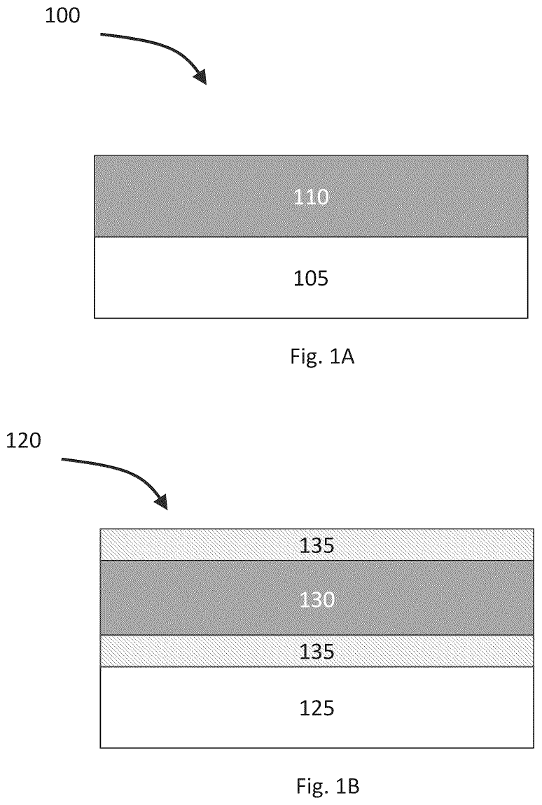

In some aspects, a 3D printed scaffold is provided, for example, a 3D printed scaffold 100, shown in FIG. 1A. In some embodiments, 3D printed scaffold 100 comprises a first layer 105 and a second layer 110. In some embodiments, the first and/or second layers comprise a plurality of substantially parallel first and/or second structures, respectively, that are substantially aligned along a first and second axis, respectively. In some embodiments, one or more radionuclides is attached to one or more exposed surfaces of the one or more structures of the one or more layers. For example, FIG. 1B shows exemplary 3D printed scaffold 120 comprising a first layer 125 comprising a radionuclide 135 and a second layer 130 comprising a radionuclide 135. The radionuclide may be adsorbed to any exposed surface within the 3D printed scaffold. For example, in some embodiments, the exposed surface is any surface of the substantially parallel first and/or second structures of the first and/or second layers, respectively.

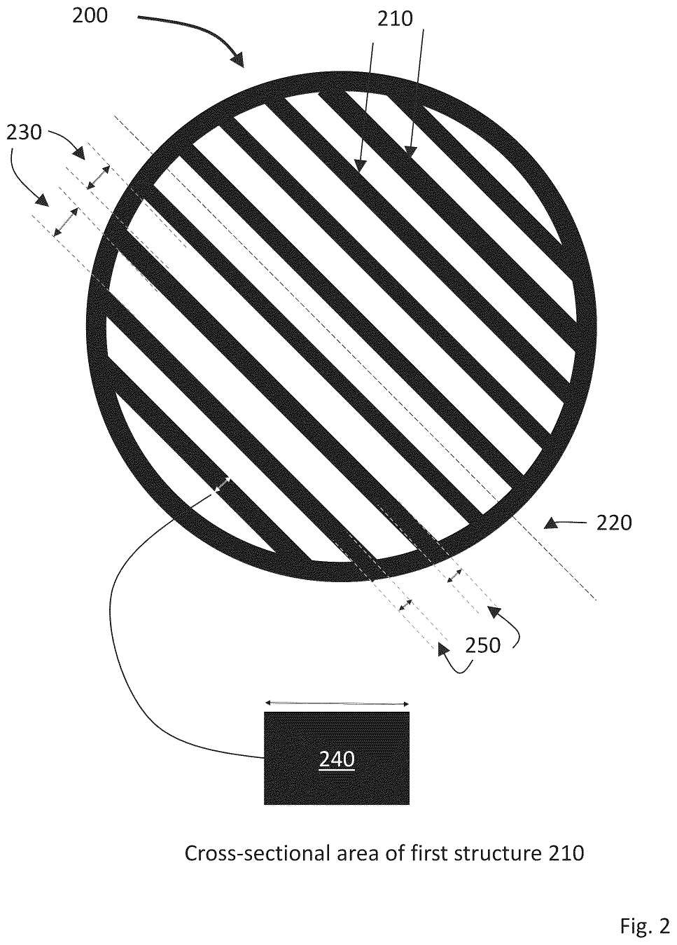

FIG. 2 shows a top view of an exemplary first layer 200 comprising a plurality of substantially parallel first structures 210. In some embodiments, the substantially parallel first structures are substantially aligned along first axis 220. As used herein the term “substantially” means “for the most part” or “essentially” aligned along a first axis; for example, substantially parallel first structures may include first structures that are mis-aligned from each other (not exactly 180 degrees apart) by less than or equal to 10%, 8%, 6%, 4%, or 2% (relative to the alignment axis). As shown illustratively in FIG. 2, the substantially parallel first structures are positioned within a single layer (e.g., a first layer). In some embodiments, the first structures have an average spacing 230 between adjacent first structures. In some embodiments, the first structures have an average width 250 and cross-sectional area 240. The skilled artisan will understand that while FIG. 2 shows a first structure with a rectangular cross-sectional geometry, the cross-sectional geometry may be any suitable cross-sectional geometry known to the skilled artisan. Exemplary cross-sectional geometries include, but are not limited to triangles, squares, rectangles, pentagons, hexagons, octagons, decagons, rhombus, parallelogram, kite, trapezium, trapezoid, or any other regular or irregular polygon known to the skilled artisan. In some embodiments, the first layer of a 3D printed scaffold may be characterized by a ratio of the average width of the first structures and the average spacing between adjacent first structures. Without wishing to be bound by any particular theory, it is generally believed that maximizing this ratio increases the available exposed surface for adsorption of the radionuclide (e.g., parent radionuclide), which in turn, increases the overall production of the desired target radionuclide.

Adjacent first structures may be configured to have any suitable average spacings between the structures. In some embodiments, the average spacing between adjacent first structures (e.g., within a first layer) is greater than or equal to 0.1 mm, greater than or equal to 0.2 mm, greater than or equal to 0.3 mm, greater than or equal to 0.4 mm, or greater than or equal to 0.5 mm. In some embodiments, the average spacing between adjacent first structures is less than or equal to 0.5 mm, less than or equal to 0.4 mm, less than or equal to 0.3 mm, less than or equal to 0.2 mm, or less than or equal to 0.1 mm. Combinations of the above-referenced ranges are also possible. For example, in some embodiments, the average spacing between substantially parallel first structures is greater than or equal to 0.1 mm and less than or equal to 0.5 mm. Other ranges are also possible.

A first structure within a plurality of substantially parallel first structures may have any suitable cross-sectional area. In some embodiments the average cross-sectional diameter of the first structure within the plurality of substantially parallel first structures (e.g., within a first layer) is greater than or equal to 20 mm², greater than or equal to 40 mm², greater than or equal to 80 mm², greater than or equal to 100 mm², greater than or equal to 200 mm², greater than or equal to 400 mm², greater than or equal to 800 mm², greater than or equal to 1000 mm², greater than or equal to 1200 mm², greater than or equal to 1400 mm², greater than or equal to 1600 mm², greater than or equal to 1800 mm², or greater than or equal to 2000 mm². In some embodiments, the average cross-sectional area of a first structure within a plurality of substantially parallel first structures is less than or equal to 2000 mm², less than or equal to 1800 mm², less than or equal to 1600 mm², less than or equal to 1400 mm², less than or equal to 1200 mm², less than or equal to 1000 mm², less than or equal to 800 mm², less than or equal to 400 mm², less than or equal to 200 mm², less than or equal to 100 mm² for, less than or equal to 80 mm², less than or equal to 40 mm², or less than or equal to 20 mm². Combinations of the above-referenced ranges are also possible. For example, in some embodiments the average cross-sectional area of the first structure within a plurality of substantially parallel structures is greater than or equal to 20 mm² and less than or equal to 2000 mm². Other ranges are also possible.

A first structure within a plurality of substantially parallel first structures may have any suitable width. In some embodiments, the average width of the first structure within a plurality of substantially parallel first structures (e.g., within a first layer) is greater than or equal to 5 mm, greater than or equal to 10 mm, greater than or equal to 15 mm, greater than or equal to 20 mm, greater than or equal to 25 mm, greater than or equal to 30 mm, greater than or equal to 35 mm, greater than or equal to 40 mm, greater than or equal to 45 mm, or greater than or equal to 50 mm. In some embodiments, the average width of the first structure within a plurality of substantially parallel first structures is less than or equal to 50 mm, less than or equal to 45 mm, less than or equal to 40 mm, less than or equal to 35 mm, less than or equal to 30 mm, less than or equal to 25 mm, less than or equal to 20 mm, less than or equal to 15 mm, less than or equal to 10 mm, or less than or equal to 5 mm. Combinations of the above-referenced ranges are also possible. For example, in some embodiments the average width of the first structure within a plurality of substantially parallel first structures is greater than or equal to 5 mm and less than or equal to 50 mm. Other ranges are also possible.

The first structures may be configured to have any suitable ratio of the average width of a first structure to the average spacing between adjacent first structures. In some embodiments the ratio of the average width of the first structure and the average spacing between adjacent first structures (e.g., within a first layer) is greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, or greater than or equal to 100. In some embodiments the ratio of the average width of the first structure and the average spacing between adjacent first structures is less than or equal to 100, less than or equal to 90, less than or equal to 80, less than or equal to 70, less than or equal to 60, less than or equal to 50, less than or equal to 40, less than or equal to 30, less than or equal to 20, and less than or equal to 10. Combinations of the above-referenced ranges are also possible. For example, in some embodiments the ratio of the average width of the first structure and the average spacing between adjacent first structures is greater than or equal to 10 and less than or equal to 100. Other ranges are also possible.

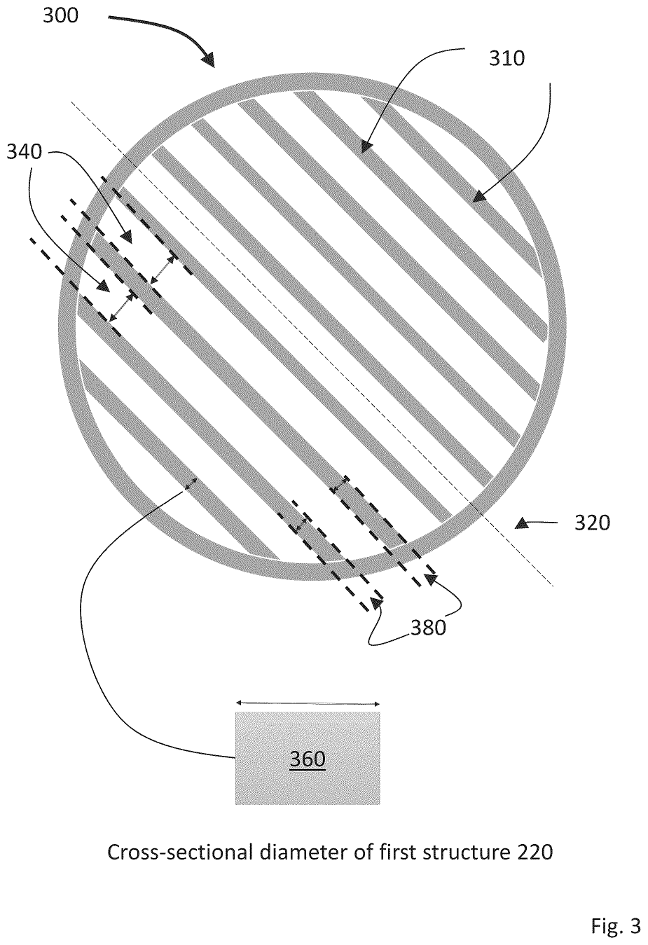

FIG. 3 shows a top view of an exemplary second layer 300 comprising a plurality of substantially parallel second structures 310. In some embodiments, the substantially parallel first structures are substantially aligned along first axis 320. As used herein the term “substantially” means “for the most part” or “essentially” aligned along a second axis; for example, substantially parallel second structures may include second structures that are mis-aligned from each other (not exactly 180 degrees apart) by less than or equal to 10%, 8%, 6%, 4%, or 2% (relative to the alignment axis). As shown illustratively in FIG. 3, the substantially parallel second structures are positioned within a single layer (e.g., a second layer). In some embodiments, the second structures have an average spacing 340 between adjacent second structures. In some embodiments, the second structures have an average width 380 and cross-sectional area 360. The skilled artisan will understand that while FIG. 3 shows a second structure with rectangular cross-sectional geometry, the cross-sectional geometry may be any suitable cross-sectional geometry known to the skilled artisan. Exemplary cross-sectional geometries include, but are not limited to triangles, squares, rectangles, pentagons, hexagons, octagons, decagons, rhombus, parallelogram, kite, trapezium, trapezoid, or any other regular or irregular polygon known to the skilled artisan. In some embodiments, the second layer of a 3D printed scaffold may be characterized by a ratio of the average width of the second structures and the average spacing between adjacent second structures. Without wishing to be bound by any particular theory, it is generally believed that maximizing this ratio increases the available exposed surface for adsorption of the radionuclide (e.g., parent radionuclide), which in turn, increases the overall production of the desired target radionuclide.

Adjacent second structures may be configured to have any suitable average spacings between the structures. In some embodiments, the average spacing between adjacent second structures (e.g., within a second layer) is greater than or equal to 0.1 mm, greater than or equal to 0.2 mm, greater than or equal to 0.3 mm, greater than or equal to 0.4 mm, or greater than or equal to 0.5 mm. In some embodiments, the average spacing between adjacent second structures is less than or equal to 0.5 mm, less than or equal to 0.4 mm, less than or equal to 0.3 mm, less than or equal to 0.2 mm, or less than or equal to 0.1 mm. Combinations of the above-referenced ranges are also possible. For example, in some embodiments, the average spacing between substantially parallel second structures is greater than or equal to 0.1 mm and less than or equal to 0.5 mm. Other ranges are also possible.

A second structure within a plurality of substantially parallel second structures may have any suitable cross-sectional area. In some embodiments the average cross-sectional diameter of the second structure within the plurality of substantially parallel second structures (e.g., within a second layer) is greater than or equal to 20 mm², greater than or equal to 40 mm², greater than or equal to 80 mm², greater than or equal to 100 mm², greater than or equal to 200 mm², greater than or equal to 400 mm², greater than or equal to 800 mm², greater than or equal to 1000 mm², greater than or equal to 1200 mm², greater than or equal to 1400 mm², greater than or equal to 1600 mm², greater than or equal to 1800 mm², or greater than or equal to 2000 mm². In some embodiments, the average cross-sectional area of a second structure within a plurality of substantially parallel second structures is less than or equal to 2000 mm², less than or equal to 1800 mm², less than or equal to 1600 mm², less than or equal to 1400 mm², less than or equal to 1200 mm², less than or equal to 1000 mm², less than or equal to 800 mm², less than or equal to 400 mm², less than or equal to 200 mm², less than or equal to 100 mm² for, less than or equal to 80 mm², less than or equal to 40 mm², or less than or equal to 20 mm². Combinations of the above-referenced ranges are also possible. For example, in some embodiments the average cross-sectional area of the second structure within a plurality of substantially parallel structures is greater than or equal to 20 mm² and less than or equal to 2000 mm². Other ranges are also possible.

A second structure within a plurality of substantially parallel second structures may have any suitable width. In some embodiments, the average width of the second structure within a plurality of substantially parallel second structures (e.g., within a first layer) is greater than or equal to 5 mm, greater than or equal to 10 mm, greater than or equal to 15 mm, greater than or equal to 20 mm, greater than or equal to 25 mm, greater than or equal to 30 mm, greater than or equal to 35 mm, greater than or equal to 40 mm, greater than or equal to 45 mm, or greater than or equal to 50 mm. In some embodiments, the average width of the second structure within a plurality of substantially parallel second structures is less than or equal to 50 mm, less than or equal to 45 mm, less than or equal to 40 mm, less than or equal to 35 mm, less than or equal to 30 mm, less than or equal to 25 mm, less than or equal to 20 mm, less than or equal to 15 mm, less than or equal to 10 mm, or less than or equal to 5 mm. Combinations of the above-referenced ranges are also possible. For example, in some embodiments the average width of the second structure within a plurality of substantially parallel second structures is greater than or equal to 5 mm and less than or equal to 50 mm. Other ranges are also possible.

The second structures may be configured to have any suitable ratio of the average width of a second structure to the average spacing between adjacent second structures. In some embodiments the ratio of the average width of the second structure and the average spacing between adjacent second structures (e.g., within a second layer) is greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, or greater than or equal to 100. In some embodiments the ratio of the average width of the second structure and the average spacing between adjacent second structures is less than or equal to 100, less than or equal to 90, less than or equal to 80, less than or equal to 70, less than or equal to 60, less than or equal to 50, less than or equal to 40, less than or equal to 30, less than or equal to 20, and less than or equal to 10. Combinations of the above-referenced ranges are also possible. For example, in some embodiments the ratio of the average width of the second structure and the average spacing between adjacent second structures is greater than or equal to 10 and less than or equal to 100. Other ranges are also possible.

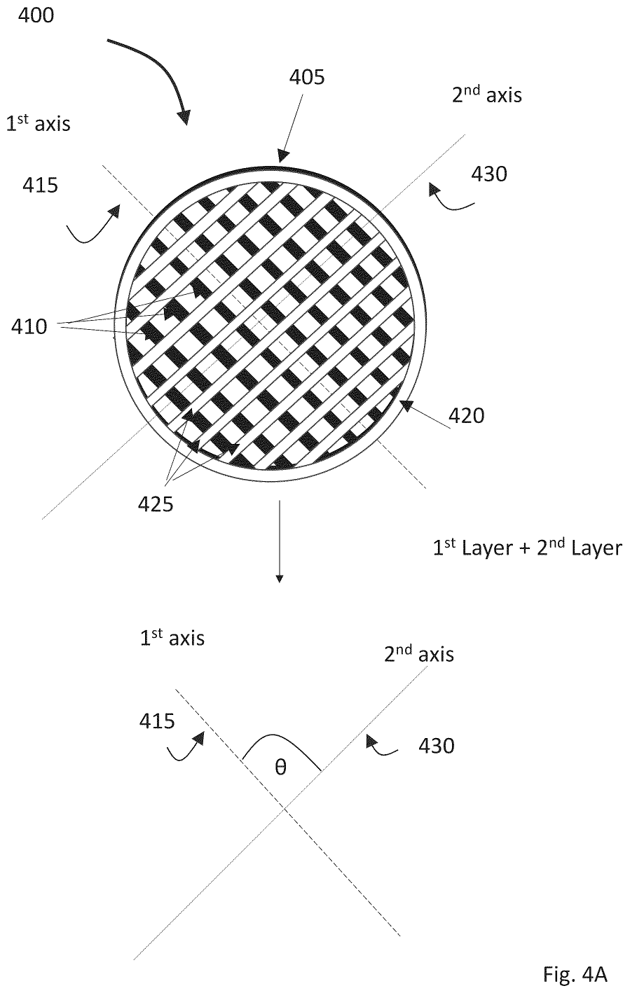

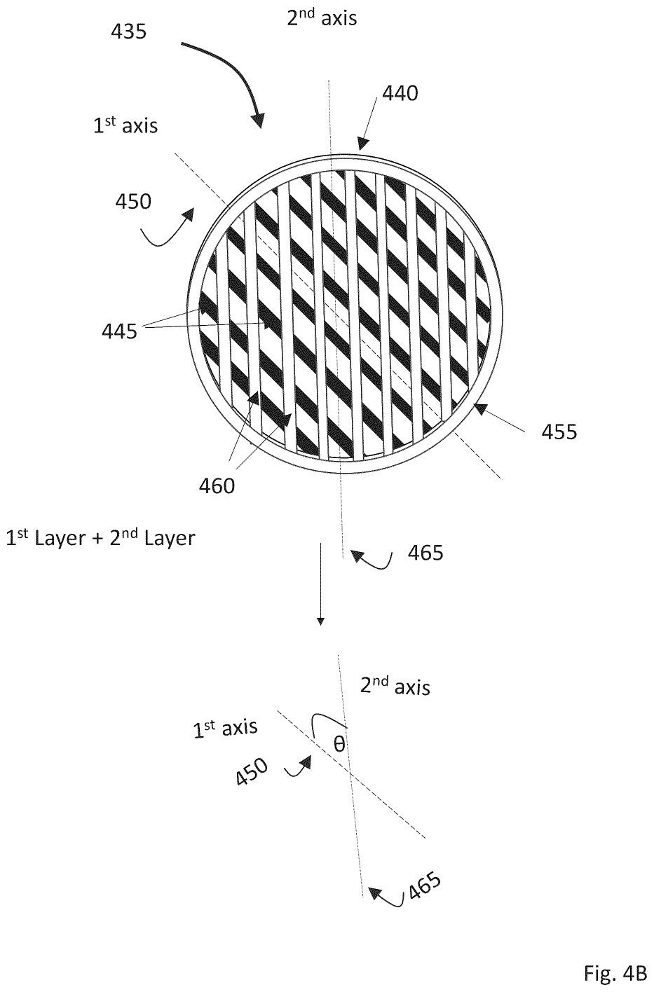

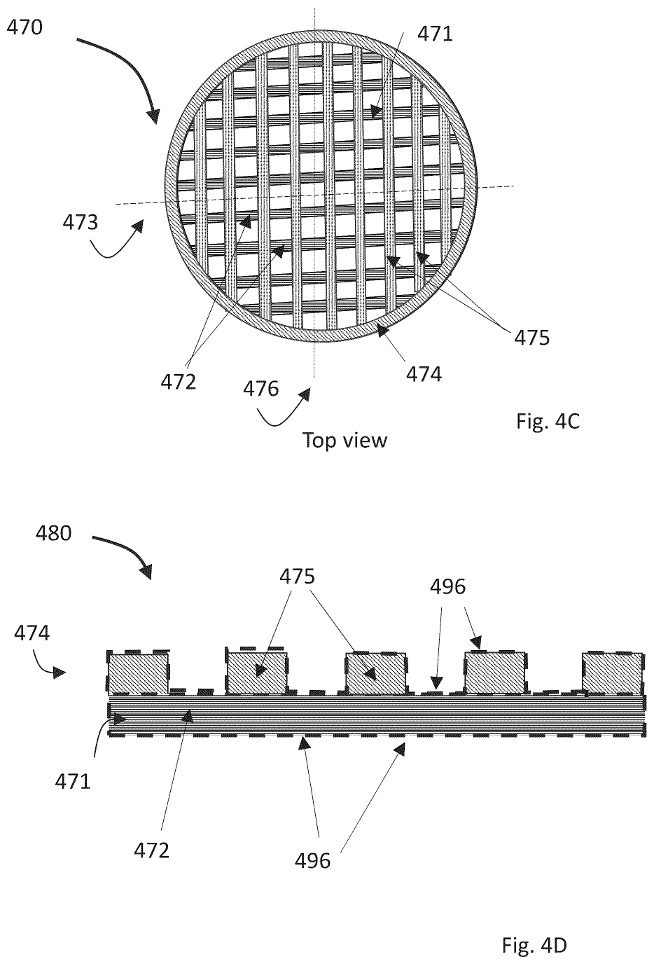

FIG. 4A shows a top view of an exemplary 3D printed scaffold 400, comprising a first layer 405 comprising a plurality of substantially parallel first structures 410 (depicted as black lines) aligned along first axis 415 and a second layer 420 comprising a plurality of substantially parallel second structures 425 (depicted as white lines) aligned along second axis 430. In some embodiments, an angle theta, formed between the first axis and second axis, is between 0 and 180 degrees. As an example, FIG. 4A shows an exemplary embodiment wherein the angle theta between the first and second axis is about 90 degrees. Other arrangements are also possible. For example, FIG. 4B shows a top view of an exemplary 3D printed scaffold 435 comprising a first layer 440 comprising a plurality of substantially parallel first structures 445 (depicted as black lines) aligned along first axis 450 and a second layer 455 comprising a plurality of substantially parallel second structures 460 (depicted as white lines) aligned along second axis 465, wherein the angle formed between the first and second axis is greater than 0 degrees and less than 90 degrees. Other ranges are also possible. For example, in some embodiments the angle formed between the first axis and the second axis is greater than 5 degrees and less than 80 degrees, greater than 10 degrees and less than 70 degrees, greater than 20 degrees and less than 60 degrees, or greater than 30 degrees and less than 50 degrees. In some embodiments, the angle formed between the first axis and second axis is greater than or equal to 0 degrees, greater than or equal to 10 degrees, greater than or equal to 20 degrees, greater than or equal to 30 degrees, greater than or equal to 40 degrees, greater than or equal to 50 degrees, greater than or equal to 60 degrees, greater than or equal to 70 degrees, greater than or equal to 80 degrees, or greater than or equal to 90 degrees. In some embodiments, the angle formed between the first axis and the second axis is less than or equal to 90 degrees, less than or equal to 80 degrees, less than or equal to 70 degrees, less than or equal to 60 degrees, less than or equal to 50 degrees, less than or equal to 40 degrees, less than or equal to 30 degrees, less than or equal to 20 degrees, less than or equal to 10 degrees, or less than or equal to 0 degrees. Combinations of the above-referenced ranges are also possible.

FIG. 4C shows a top view of an exemplary 3D printed scaffold 470 comprising a first layer 471 comprising a plurality of substantially parallel first structures 472 aligned along a first axis 473 and a second layer 474 comprising a plurality of substantially parallel second structures 475 aligned along a second axis 476. FIG. 4D shows a cross-sectional view 480 of 3D printed scaffold 470 (e.g., along first axis 473). FIG. 4D also illustrates a radionuclide 496 (e.g., a parent radionuclide) disposed on one or more exposed surfaces of the first structures of the first layer and/or one or more exposed surfaces of the second structures of the second layer of 3D printed scaffold 470, respectively. It should be noted that while radionuclide 496 is represented as a layer adjacent the first and/or second layers in FIG. 4C, the skilled artisan will understand that radionuclide (e.g., parent radionuclide) 496 may not necessarily form a layer that uniformly coats the underlying exposed surface. For example, in some embodiments, the radionuclide (e.g., parent radionuclide) 496 may be deposited as clusters or islands (e.g., periodically, randomly) on the exposed surface of the first and/or second layers. In some embodiments, the radionuclide (e.g., parent radionuclide) may form a continuous or discontinuous layer on the exposed surfaces. For example, the radionuclides may be present on the one or more exposed surfaces of the first and/or second structures of the first and/or second layers as a monolayer, a multilayer, or as discrete islands. Combinations of the above-referenced ranges are also possible, for example, the radionuclides may form discrete islands disposed on top of a mono- or multilayer.

The radionuclide may be attached to the one or more exposed surfaces of the first structure and/or the one or more exposed surfaces of the second structures using any suitable technique known to those of skill in the art. For example, in some instance, the radionuclide may be attached via adsorption. Without wishing to be bound by any particular theory, it is believed that adsorption may occur via several different mechanism, including, for example, physisorption and chemisorption. Accordingly, in some embodiments, the radionuclide is adsorbed via physisorption. As used herein, physisorption refers to adsorption mediated by van der Waals forces and is a weak intermolecular attraction that takes place below the critical temperature of the adsorbate and can result in the development of a monolayer or multilayer of the adsorbate. In other instances, the radionuclide may be adsorbed via chemisorption. As used herein, “chemisorption” refers to adsorption in which there is a chemical bond between the adsorbent (e.g., 3D printed scaffold) and the adsorbate (e.g., radionuclide). Other mechanisms of adsorption may also be possible. For example, in some embodiments, the radionuclide may be adsorbed to the structures of the 3D printed scaffold via one or more electrostatic interactions.

Other mechanisms for attaching a radionuclide are also contemplated herein. For example, in some embodiments, radionuclides may be physically entrapped within defects present on the one or more surfaces of the first and/or second structures. Alternatively, or additionally, in some embodiments, a metal chelator is used to chelate the radionuclide and the metal chelator is subsequently attached, either directly or indirectly, to the 3D printed scaffold. Any suitable metal chelator may be used to chelate the radionuclide, such as those disclosed in Yang et al., “Harnessing alpha-emitting radionuclides for therapy: radiolabeling method review.” The Journal of Nuclear Medicine Vol. 63, No. 1, 2022; and Holik et al., “The chemical scaffold of theranostic radiopharmaceuticals: radionuclide, bifunctional chelator, and pharmacokinetics modifying linker.” Molecules Vol. 27, 2022, p. 3062, both of which are incorporated herein by reference in their entirety.

In some embodiments, the radionuclide is embedded in at least a portion of the material forming the 3D printed scaffold. In other embodiments, the radionuclide is not embedded within the material forming the 3D printed scaffold, and may optionally be attached to the material by one or more methods described herein.

The radionuclide may further be mixed with one or more materials disclosed herein. For example, in some embodiments, the radionuclide is mixed with a ceramic slurry or a metallic slurry. Such slurries can be used to coat or print the 3D printed scaffolds, as contemplated herein, using any suitable technique known to one of skill in the art (e.g., such as those discussed in detail below). Without wishing to be bound by any particular theory, it is believed that 3D printed scaffolds printed with such slurries would comprise the radionuclides throughout the bulk of the scaffold. In other words, the radionuclide would be present on an exposed outer surface of a structure (e.g., a first structure) as well as within the interior of said structure (e.g., first structure). In other embodiments, a 3D printed scaffold could be coated with a material comprising both the radionuclide and another material or slurry.

In some embodiments, the slurry comprises a ceramic or a metal. However, in some cases, the slurry may further comprise one or more porogens (e.g., various polymers, salts, and/or sugar particles) or blowing agents (e.g., ammonium bicarbonate, sodium bicarbonate, or sodium borohydride, etc.) configured to introduce a plurality of pores within the one or more structures (e.g., first structure, second structure, etc.) of the 3D printed scaffolds. Without wishing to be bound by any particular theory, it is believed that heating a blowing agent above a certain temperature will cause that blowing agent to transform from a solid material into a gas. Such processes are routinely used in the art, for example, for the production of porous foams as described by Jin et al., “Recent Trend of Foaming in Polymer Processing: A Review,” Polymers (Basel), 2019; 11(6):953, which is herein incorporated by reference in its entirety. Again, without wishing to be bound by any particular theory, it is believed that the introduction of said pores within the one or more structures (e.g., a first structure) increases the exposed surface area available for adsorption of the radionuclide (e.g., for radionuclides loaded after formation of the 3D printed scaffold). Alternatively, it is believed that the inclusion of said pores within the one or more structures of a 3D printed scaffold comprising the radionuclide embedded within the bulk of one or more structures (e.g., a first structure), increases the surface concentration of exposed radionuclide (e.g., it increases the surface area of the structure thereby exposing a higher concentration of the radionuclide embedded within said structure).

The radionuclide may be attached to one or more exposed surfaces described herein (e.g., exposed surface(s) of a first structure, second structure, scaffold) in any suitable amount. In some embodiments, the attached radionuclide covers greater than or equal to 1%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the one or more exposed surfaces of the first and/or second structures. In some embodiments the attached radionuclide covers less than or equal to 100%, less than or equal to 80%, less than or equal to 60%, less than or equal to 40%, less than or equal to 20%, less than or equal to 10%, or less than or equal to 5% of the one or more exposed surfaces of the first and/or second structures. Combinations of the above-referenced ranges are also possible. For example, in some embodiments, the attached radionuclide covers greater than or equal to 50% and less than or equal to 100% of the one or more exposed surfaces of the first and/or second structures.

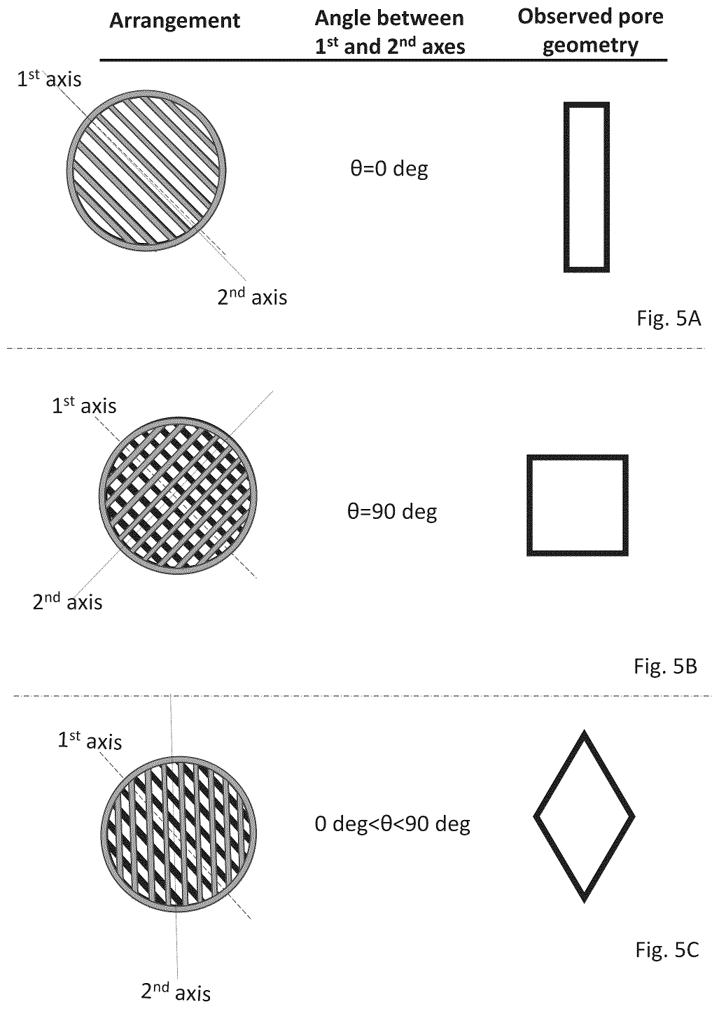

FIGS. 5A-5C show the exemplary pore shapes that may be formed in the spacings between the first and second substantially parallel structures in the first and second layers, respectively, when viewed from the “top” position for 3D printed scaffolds comprising theta values of between 0 degrees and 90 degrees. For example, in the scenario where the first axis and the second axis are substantially parallel, the observed pore geometry will be substantially rectangular (e.g., FIG. 5A). For example, if the angle between the first axis and the second axis is 90 degrees, the plurality of first structures and the plurality of second structures form a plurality of rectangular pores. In certain scenarios where the first axis and the second axis are substantially perpendicular, and the spacing between the adjacent first structures is the same as the spacing between the second structures, the observed pore geometry may resemble a square (FIG. 5B). In all other scenarios, the observed pore geometry will be some form of a polygon with four sides (e.g., a diamond shape as shown in FIG. 5C). Other pore geometries are also possible. For example, the addition of a third layer comprising plurality of substantially parallel third structures substantially aligned along a third axis, wherein the first axis, second axis, and third axis are all perpendicular to each other, or otherwise off-set from each other, would produce substantially triangular pores.

In some embodiments, a pore area formed by the intersection of the first axis and the second axis (e.g., the area within the shape listed under “Observed Pore Geometry” in FIG. 5) is between 0.1 mm² and 1 mm². In some embodiments, the pore area is greater than or equal to 0.1 mm², greater than or equal to 0.2 mm², greater than or equal to 0.3 mm², greater than or equal to 0.4 mm², greater than or equal to 0.5 mm², greater than or equal to 0.6 mm², greater than or equal to 0.7 mm², greater than or equal to 0.8 mm², greater than or equal to 0.9 mm², or greater than or equal to 1.0 mm². In some embodiments, the pore area is less than or equal to 1.0 mm², less than or equal to 0.9 mm², less than or equal to 0.8 mm², less than or equal to 0.7 mm², less than or equal to 0.6 mm², less than or equal to 0.5 mm², less than or equal to 0.4 mm², less than or equal to 0.3 mm², less than or equal to 0.2 mm², or less than or equal to 0.1 mm². Combinations of the above-referenced ranges are also possible. For example, in some embodiments, the pore area formed by the intersection of the first axis and the second axis is greater than or equal to 0.1 mm² and less than or equal to 1.0 mm².

The pores formed by the intersection of the first axis and the second axis (e.g., the area within the shape listed under “Observed Pore Geometry” in FIG. 5) may have any suitable average aspect ratio. In some embodiments, the average aspect ratio is greater than or equal to 1:1, greater than or equal to 2:1, greater than or equal to 3:1, greater than or equal to 4:1, greater than or equal to 5:1, greater than or equal to 6:1, greater than or equal to 7:1, greater than or equal to 8:1, greater than or equal to 9:1, or greater than or equal to 10:1. In some embodiments, the average aspect ratio is less than or equal to 10:1, less than or equal to 9:1, less than or equal to 8:1, less than or equal to 7:1, less than or equal to 6:1, less than or equal to 5:1, less than or equal to 4:1, less than or equal to 3:1, less than or equal to 2:1, less than or equal to 1:1. Combinations of the above-referenced ranges are also possible. For example, in some embodiments, the average aspect ratio is greater than or equal to 1:1, and less than or equal to 10:1.

As described, pores within a 3D printed scaffold comprising a radionuclide (e.g., parent radionuclide), provide a path for gaseous daughter intermediates (e.g., ²²⁰Rn), formed in the decay chain of the radionuclide (e.g., parent radionuclide), to diffuse through. This allows the gaseous intermediate to travel to a location different from the scaffold (e.g., via diffusion or active transport via an inert carrier gas) before decaying into one or more other radionuclides. In some embodiments, the gaseous daughter intermediate is transported out of the scaffold in greater than or equal to 1 second, greater than or equal to 10 seconds, greater than or equal to 20 seconds, greater than or equal to 30 seconds, greater than or equal to 40 seconds, greater than or equal to 50 seconds, greater than or equal to 60 seconds, greater than or equal to 2 minutes, or greater than or equal to 5 minutes. In some embodiments, the gaseous daughter intermediate is transported out of the scaffold in less than or equal to 5 minutes, less than or equal to 2 minutes, less than or equal to 60 seconds, less than or equal to 50 seconds, less than or equal to 40 seconds, less than or equal to 30 seconds, less than or equal to 20 seconds, less than or equal to 10 seconds, or less than or equal to 1 second. Combinations of the above ranges are also possible. For example, in some embodiments, the gaseous daughter intermediate is transported out of the scaffold in greater than or equal to 1 second and less than or equal to 5 minutes).

In some embodiments, a gaseous intermediate may decay to a more other radionuclides prior to diffusing out of a 3D printed scaffold. For example, in some cases, radionuclide (e.g., parent radionuclide)²²⁴Ra within 3D printed scaffold comprising radionuclide (e.g., parent radionuclide)²²⁴Ra undergoes radioactive decay to produce ²²⁰Rn which subsequently undergoes radioactive decay to yield ²¹⁶Powhich further decays to ²¹²Pb (which is stable for ˜10 hours). In some embodiments, the one or more radionuclides is deposited on one or more exposed surfaces of the 3D printed scaffold. In some cases, the surface area. In some embodiments, the radionuclide covers greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, or greater than or equal to 10% of the one or more exposed surfaces of the 3D printed scaffold. In some embodiments the radionuclide covers less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, or less than or equal to 1% of the one or more exposed surfaces of the 3D printed scaffolds. Combinations of the above-referenced ranges are also possible. For example, in some embodiments, the radionuclide covers greater than or equal to 1% and less than or equal to 10% of the one or more exposed surfaces of the 3D printed scaffold.

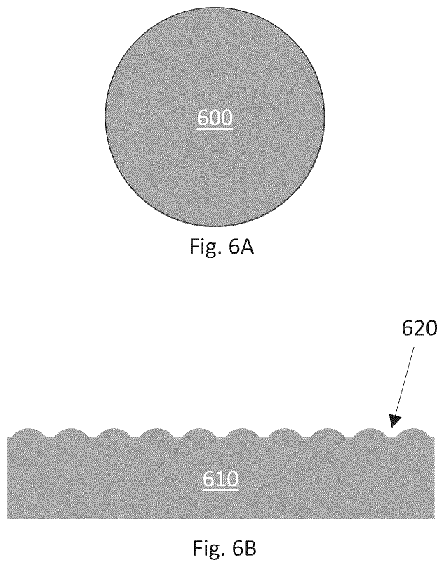

In some embodiments, the 3D printed scaffolds disclosed herein comprise one or more nonporous layers, such as a nonporous layer 600 shown as a top view in FIG. 6A. In some embodiments, the nonporous layer is a first layer, a second layer, or a side layer. In some embodiments, one or more surfaces of the first layer, second layer or side layer are a nonporous layer. In some embodiments, the nonporous layer comprises a plurality of surface features in the layer. For instance, the surface features may be in the form of indentations and/or undulations. The surface features may include, for example, grooves and/or channels 620, as shown illustratively in nonporous layer 610 in FIG. 6B.

The shape of the plurality of surface features (e.g., grooves and/or channels) may be any suitable shape known to the skill artisan. Exemplary shapes include, but are not limited to, “U” shaped grooves, “V” shaped grooves, square bottom grooves, and the like. The feature depth may be between 0.1 mm² and 1 mm². In some embodiments, the feature depth is greater than or equal to 0.1 mm², greater than or equal to 0.2 mm², greater than or equal to 0.3 mm², greater than or equal to 0.4 mm², greater than or equal to 0.5 mm², greater than or equal to 0.6 mm², greater than or equal to 0.7 mm², greater than or equal to 0.8 mm², greater than or equal to 0.9 mm², or greater than or equal to 1.0 mm². In some embodiments, the feature depth is less than or equal to 1.0 mm², less than or equal to 0.9 mm², less than or equal to 0.8 mm², less than or equal to 0.7 mm², less than or equal to 0.6 mm², less than or equal to 0.5 mm², less than or equal to 0.4 mm², less than or equal to 0.3 mm², less than or equal to 0.2 mm², or less than or equal to 0.1 mm², Combinations of the above-referenced ranges are also possible. For example, in some embodiments, the feature depth is greater than or equal to 0.1 mm² and less than or equal to 1.0 mm².

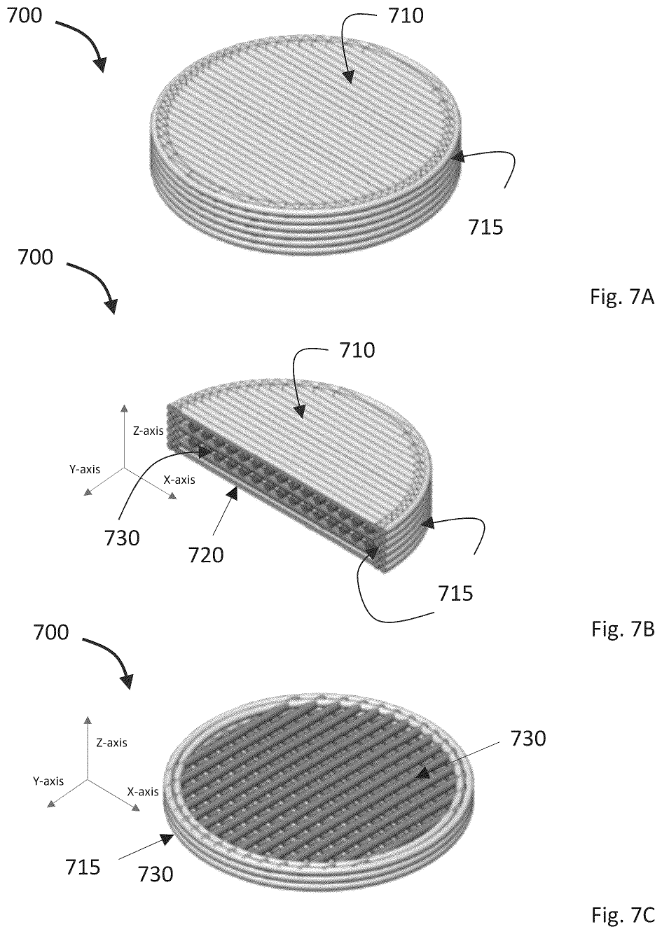

In some embodiments, all or portions of a 3D printed scaffold may be enclosed (partially or fully) with one or more nonporous layers. FIGS. 7A-7C show a top view (FIG. 7A), a cross-sectional view along the x-axis (FIG. 7B) and a cross-sectional view along the y-axis (FIG. 7C) of exemplary 3D printed scaffold 700 comprising nonporous bottom layer 720, nonporous side layer 715, nonporous top layer 710, and first and/or second layer 730. In some embodiments, first and/or second layer 730 of the 3D printed scaffold, along with a third layer and a fourth layer, are positioned within the nonporous bottom layer, the porous top layer, and the nonporous side layers.

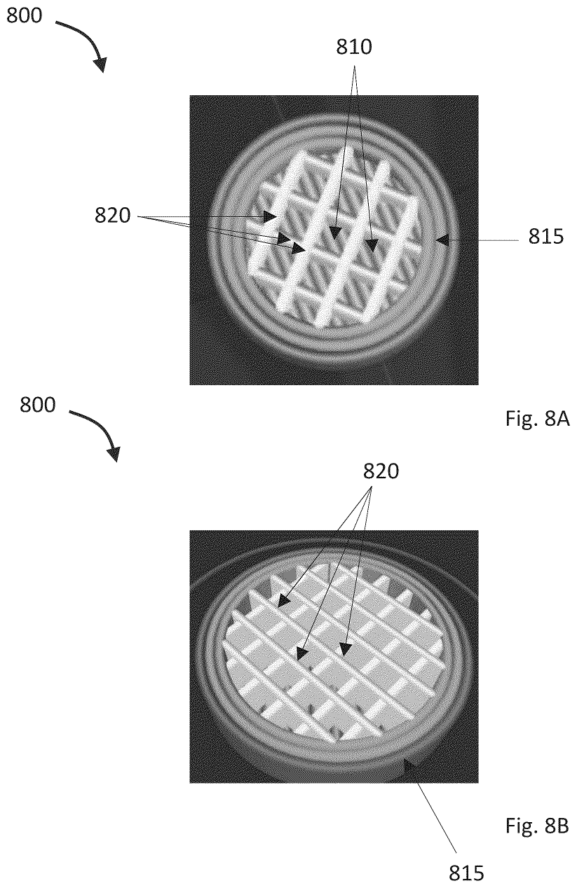

FIGS. 8A-8B show a top view of exemplary 3D printed scaffold 800 comprising a nonporous bottom layer including ridges 810 (which is visible through porous layer 820), a nonporous side layer 815 and a second, porous layer 820. The first and second layers of the 3D printed scaffold may be positioned within a third, porous (side) layer 816 and a fourth, porous (side) layer 818, each of which may be positioned within nonporous side layer 815. In other embodiments, the scaffold does not include a nonporous bottom layer or nonporous top layer, but includes a nonporous side layer. Other configurations are also possible.

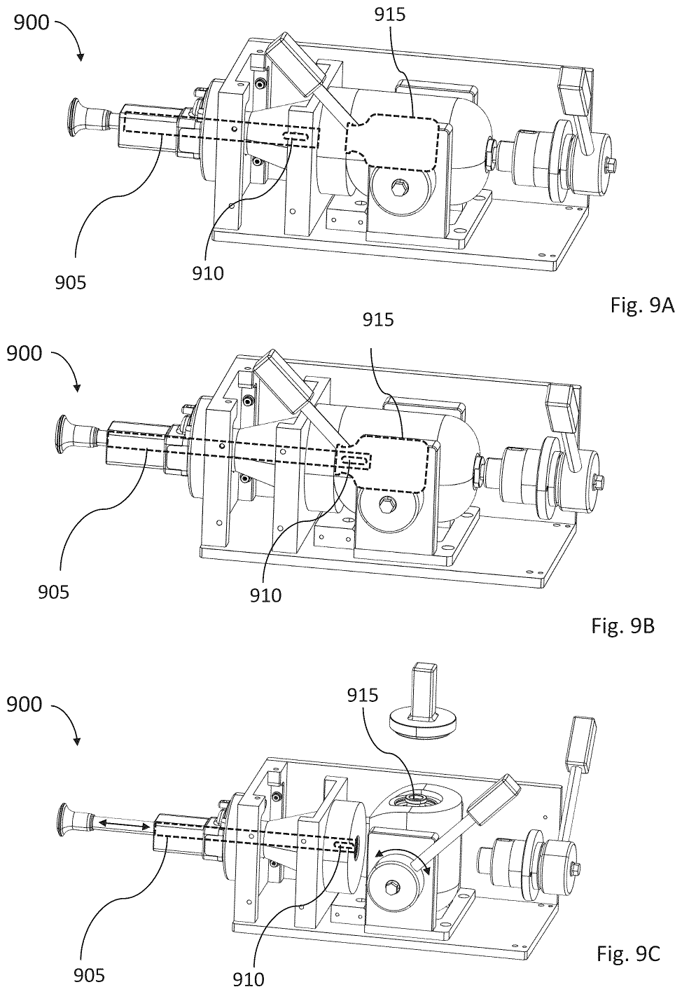

FIG. 9A shows a side view of an exemplary radionuclide generator 900 comprising a source module 905 that comprises radionuclide-loaded 3D printed scaffold 910 (including a parent nucleotide such as ²²⁴Ra or ²²⁸Th) at a first location, a location different than collection vessel 915. FIG. 9B shows a side view of an exemplary radionuclide generator 900 comprising source module 905 comprising radionuclide-loaded 3D printed scaffold 910 at a second location, e.g., within collection vessel 915. In some embodiments, at least a portion of the radionuclide loaded within 3D printed scaffold 910 decays to ²²⁰Rn at the first location and is collected at the second location within collection vessel 915. In some embodiments, at least some of such decay occurs at the second location. FIG. 9C shows a side view of an exemplary radionuclide generator 900 comprising source module 905 that comprises radionuclide-loaded 3D printed scaffold 910 and collection vessel 915 comprising ²²⁰Rn and/or one of its decay products (e.g., ²¹²Pb).

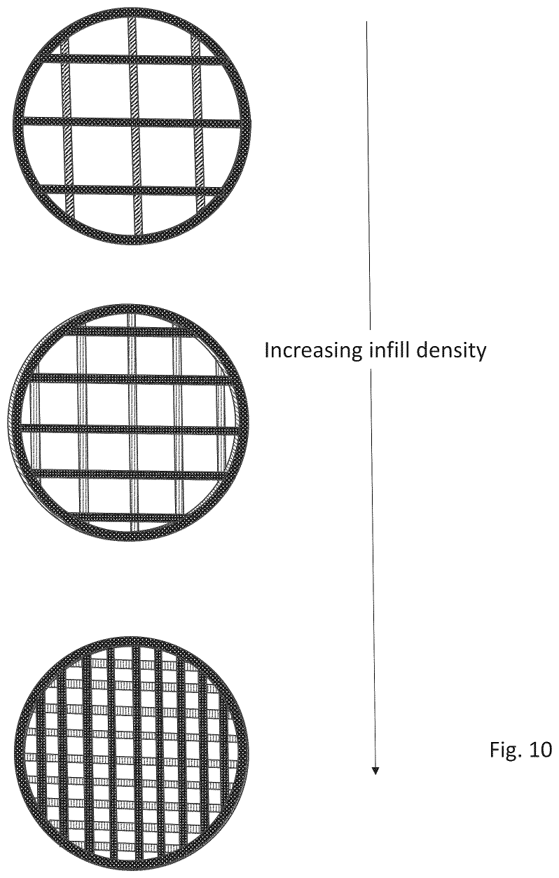

FIG. 10 shows a top view of an exemplary 3D printed scaffold comprising a first layer comprising a plurality of substantially parallel first structures aligned along a first axis and a second layer comprising a plurality of substantially parallel second structures aligned along a second axis with varying infill densities. The infill density, described elsewhere herein, can be varied by tuning the width of parallel structures and the distance between adjacent parallel structures. For example, for a given width, decreasing the distance between adjacent parallel structures will increase the infill density (and vice versa).

FIG. 11 shows an exemplary decay chain for thorium-232 (²³²Th). For example, in some embodiments, the 3D printed scaffold is loaded with a radionuclide in the ²³²Th decay chain (e.g., parent radionuclide), such as, for example, Radium-224 (²²⁴Rn). The ²²⁴Rn will spontaneously decay to produce an alpha particle and daughter radionuclide, e.g., Radon-220 (²²⁰Ra). Daughter radionuclide ²²⁰Ra, which is a gas, may diffuse out of and away from the 3D printed scaffold to a location that is different from the 3D printed scaffold. Once at the new location, the first daughter radionuclide, e.g., ²²⁰Ra, may further decay to produce an alpha particle and a second daughter radionuclide, e.g., Polonium-216 (²¹⁶Po) (daughter radionuclide of ²²⁰Ra and granddaughter radionuclide of ²²⁴Rn). Because ²¹⁶Po is an unstable isotope, with a half-life of only 0.14 seconds, it decays to a third daughter radionuclide, e.g., Lead-212 (²¹²Pb), near instantaneously (daughter of ²¹⁶Po, granddaughter of ²²⁰Ra, and great granddaughter of ²²⁴Rn).

In some embodiments, any one of the 3D printed scaffold disclosed herein comprise one or more additional layers. For example, in some embodiments, the 3D printed scaffold comprises a third layer, a fourth layer, a fifth layer, a sixth layer, a seventh layer, an eighth layer, a ninth layer, a tenth layer, and so on. In some embodiments, the one or more additional layers comprise a plurality of substantially parallel structures (e.g., third parallel structures, fourth parallel structures, fourth parallel structure, fifth parallel structures, sixth parallel structures, seventh parallel structures, eighth parallel structure, ninth parallel structures, tenth parallel structures, etc.) aligned along an axis (e.g., a third axis, a fourth axis, a fifth axis, a sixth axis, a seventh axis, an eighth axis, a ninth axis, a tenth axis, etc.). When two or more substantially parallel structures are present in a layer, the amount of each type of substantially parallel structure present in the layer (e.g., average cross-sectional area of first and/or second structures, average width of first and/or second structures, angle between first axis and second axis, or the ratio between the average width of the first and/or second structure and the average spacing between adjacent first and/or second structures, among others) may independently be in one or more of the above-referenced ranges and/or all of the substantially parallel structures together may be present in an amount in one or more of the above-referenced ranges. When two or more layers (e.g., a third layer, a fourth layer, a fifth layer, and so on) comprising the substantially parallel structures (e.g., average cross-sectional area of first and/or second structures, average width of first and/or second structures, angle between first axis and second axis, or the ratio between the average width of the first and/or second structure and the average spacing between adjacent first and/or second structures, among others) are present, the preceding sentence may be independently true for each such additional layer (e.g., a third layer, a fourth layer, a fifth layer, and so on).

In some embodiments, one or more structures of a layer (e.g., a first structure of a first layer, a second structure of a second layer, etc.,) has a particular range of surface roughness.

In some embodiments, one or more structures of a layer (e.g., a first structure of a first layer, a second structure of a second layer, etc.) is relatively smooth. The smoothness or roughness of a layer may be characterized in a variety of manners. Suitable parameters that may be employed to characterize the roughness of a nonporous layer and suitable values of such parameters are described in further detail below. Some of the techniques below may be employed with reference to cross-section of the one or more structures of the layer and it should be understood that some of the one or more structures of a layer may comprise at least one cross-section having one or more of the properties described below, that some of the one or more structures of a layer may be made up exclusively of cross-sections having one or more of the properties described below, and that some of the one or more structures of a layer may have a morphology such that a majority of the cross-sections have one or more of the properties below (e.g., at least 50% of the cross-sections, at least 75% of the cross-sections, at least 90% of the cross-sections, at least 95% of the cross-sections, or at least 99% of the cross-sections).

One example of a parameter that may be employed to characterize the roughness of one or more structures of a layer (e.g., a first structure of a first layer, a second structure of a second layer, etc.,) is Ra, which is the arithmetic average deviation across the cross-section of the height of the one or more structures of a layer from the mean line of the cross section (i.e., the line which is parallel to the surface and divides the cross-section such that the area between the surface topography and the line therebeneath is equivalent to the area between the surface topography and the line thereabove). In some embodiments, the one or more structures of a layer has a value of Ra of less than or equal to 1.5 microns, less than or equal to 1.25 microns, less than or equal to 1 micron, less than or equal to 0.75 microns, less than or equal to 0.5 microns, less than or equal to 0.25 microns, less than or equal to 0.2 microns, less than or equal to 0.18 microns, less than or equal to 0.15 microns, less than or equal to 0.125 microns, less than or equal to 0.1 micron, or less than or equal to 0.075 microns. In some embodiments, the one or more structures of a layer has a value of Ra of greater than or equal to 0.05 microns, greater than or equal to 0.075 microns, greater than or equal to 0.1 micron, greater than or equal to 0.125 microns, greater than or equal to 0.15 microns, greater than or equal to 0.18 microns, greater than or equal to 0.2 microns, greater than or equal to 0.25 microns, greater than or equal to 0.5 microns, greater than or equal to 0.75 microns, greater than or equal to 1 micron, or greater than or equal to 1.25 microns. Combinations of the above-referenced ranges are also possible (e.g., less than or equal to 1.5 microns and greater than or equal to 0.05 microns, or less than or equal to 1.5 microns and greater than or equal to 0.18 microns). Other ranges are also possible.

Advantageously, introducing surface features and/or increasing surface roughness in one or more structures of a layer of the scaffold may increase the surface area of adsorption by a radionuclide (e.g., parent radionuclide).

Those of skill in the art will appreciate that one or more structures of a layer (e.g., a first structure of a first layer, a second structure of a second layer, etc.,) may comprise a configuration other than those described above. For example, in some embodiments, the layer (e.g., a first layer) comprises a plurality of structures (e.g., first structures), wherein the plurality of structures are not substantially parallel (e.g., the structures are curved) and/or not aligned along an axis (e.g., a first axis). Other configurations are also possible and may comprise any suitable configuration known to the skilled artisan (e.g., haring bone, concentric circles, etc.).

The 3D printed scaffolds disclosed herein may comprise one or more configurations comprising one or more of any one of the layers described herein. For example, in some embodiments the configuration comprises a “closed top and bottom” geometry comprising one or more layers positioned within a nonporous bottom layer, a nonporous top layer, and a nonporous side layer (e.g., FIG. 7A-7C). In some embodiments, the configuration comprises an “open top” geometry comprising one or more layers positioned with a nonporous bottom layer and a nonporous side layer (e.g., the one or more layers is exposed on the top side, FIG. 8A-8B). In some embodiments, the configuration comprises a “mesh” geometry comprises one or more layers where the observed pore geometry created by the one or more layers form a mesh structure when viewed from a top view (e.g., FIGS. 4-5).

The 3D printed scaffolds may comprise any suitable amount of infill density. As used herein, the term “infill density” refers to the amount of material (e.g., zirconia and/or quartz) within an outer boundary, and can refer to the volume of solid structures (e.g., quartz or zirconia) forming the layers of the scaffold as a fraction of the total volume of the scaffold as defined by its outermost boundary. The infill density can be varied by tuning the width of parallel structures and the distance between adjacent parallel structures (FIG. 10). For example, for a given width, decreasing the distance between adjacent parallel structures will increase the infill density (and vice versa). Accordingly, in some embodiments, the infill density is greater than or equal to 10%, greater than or equal to 20%, greater than or equal to 30%, greater than or equal to 40%, greater than or equal to 50%, greater than or equal to 60%, greater than or equal to 70%, greater than or equal to 80%, or greater than or equal to 90%. In some embodiments the infill density is less than 100%, less than or equal to 90%, less than or equal to 80%, less than or equal to 70%, less than or equal to 60%, less than or equal to 50%, less than or equal to 40%, less than or equal to 30%, less than or equal to 20%, or less than or equal to 10%. Combinations of the above-referenced ranges are also possible. For example, in some embodiments the infill density is greater than or equal to 10% and less than or equal to 90%. Other ranges are also possible.

The 3D printed scaffolds disclosed herein may comprise one or more materials suitable for use with a radionuclide (e.g., parent radionuclide). As described elsewhere herein, radionuclides are recognized compounds known to emit high energy particles (e.g., α-particles, β⁻ particles, β⁺ particles, γ-particles, Auger electrons, etc.) as the radionuclide decays (e.g., to produce a daughter radionuclide). Without wishing to be bound by any particular theory, it is believed that this process can damage the properties (e.g., mechanical properties) of the one or more materials or structures the radionuclide is associated with, causing the one or more materials or structures to degrade and/or breakdown over time (e.g., via a process known as radiolytic degradation). The rate of degradation and the degree of degradation of the one or more materials depends on the type of particle being emitted and the susceptibility of the material to radiolytic decay via said emitted particle.

The 3D printed scaffolds may include one or more ceramics and/or metals. Suitable materials may include ones that are relatively resistant to degradation under the influence of high-energy radiation, which can otherwise cause the material to lose their mechanical, electrical, chemical, and/or optical properties. For example, it is believed that exposure of certain ceramic materials to ionizing radiation affects the crystal lattice of the material by displacement of atoms within the lattice, transformation of atoms to different species by nuclear reactions, and ionization and electronic excitation. Over time, these effects may cause the ceramic lattice to break down. Metals are also susceptible to radiation damage. For example, the collision between a high energy particle and a metal atom can create vacancies within the metal lattice structure (e.g., similar to ceramics), creating point defects within the metal lattice that can alter the metal's structural, electronic, and/or thermal properties. Though these effects may not be completely avoided when being exposed to high-energy radiation, these effects may be mitigated by choosing appropriate materials, configurations, and/or processing conditions. A review of radiation effects on structural materials has been reported by Schonbacher and Tavlet, “Radiation effects on structural materials for high-energy particle accelerators and detectors,” International Workshop on Advanced Materials for High-precision Detectors, DOI: e-proceedings: 10.5170/CERN-1994-007, which is incorporated herein by reference in its entirety.

Additionally, or alternatively, a 3D printed scaffold disclosed herein may comprise one or more materials suitable for use with an acid. For instance, in some cases the 3D printed scaffold may be submerged in an acidic solution (e.g., a nitric acid solution) to facilitate loading of the radionuclide into the 3D printed scaffolds and onto one or more exposed structures of the 3D printed scaffold. In one particular embodiment, for example, the 3D printed scaffold comprises a thorium (IV) nitrate salt that is formed via the reaction of thorium(IV) hydroxide and nitric acid (HNO₃):

Th(OH)₄+4HNO₃+3H₂O→Th(NO₃)₄·5H₂0

This salt may be formed on a surface of the 3D printed scaffold by exposing the scaffold to a mixture of thorium(IV) hydroxide and nitric acid. As described in more detail below, any suitable acid (e.g., nitric acid, sulfuric acid, etc.) at any suitable concentration (e.g., 0.1 M, 0.5 M, etc.) may be used to prepare the radionuclide for loading onto the 3D printed scaffold.

In some embodiments, the 3D printed scaffolds comprise an inorganic material. In some embodiments, the inorganic material is a ceramic. In some embodiments, the inorganic material comprises a metal. Exemplary metals include, but are not limited to, zirconium, yttrium, manganese, chromium, molybdenum, vanadium, iron, niobium, cobalt, aluminum, titanium, and nickel. In some embodiments, the inorganic material comprises a non-metal. Exemplary non-metals include, but are not limited to oxygen, sulfur, phosphorus, quartz, and silicon. In some embodiments, the 3D printed scaffolds comprises stainless steel (e.g., H13). In some embodiments, the 3D printed scaffold comprises fused silica (e.g., non-crystalline silica glass). In other embodiments, the 3D printed scaffolds comprises Inconel.

In some embodiments, the percent by mass of metal in the one or more materials is greater than or equal to 0.1 wt %, greater than or equal to 1 wt %, greater than or equal to 5 wt %, greater than or equal to 10 wt %, greater than or equal to 15 wt %, greater than or equal to 20 wt %, greater than or equal to 25 wt %, greater than or equal to 30 wt %, greater than or equal to 35 wt %, greater than or equal to 40 wt %, greater than or equal to 45 wt %, greater than or equal to 50 wt %, greater than or equal to 55 wt %, greater than or equal to 60 wt %, greater than or equal to 65 wt %, greater than or equal to 70 wt %, greater than or equal to 75 wt %, greater than or equal to 80 wt %, greater than or equal to 85 wt %, greater than or equal to 95 wt %, or greater than or equal to 95 wt %. In some embodiments, the percent by mass of metal in the one or more materials is less than or equal to 95 wt %, less than or equal to 90 wt %, less than or equal to 85 wt %, less than or equal to 80 wt %, less than or equal to 75 wt %, less than or equal to 70 wt %, less than or equal to 65 wt %, less than or equal to 60 wt %, less than or equal to 55 wt %, less than or equal to 50 wt %, less than or equal to 45 wt %, less than or equal to 40 wt %, less than or equal to 35 wt %, less than or equal to 30 wt %, less than or equal to 25 wt %, less than or equal to 20 wt %, less than or equal to 15 wt %, less than or equal to 10 wt %, less than or equal to 5 wt %, less than or equal to 1 wt %, or less than or equal to 0.1 wt %. Combinations of the above-referenced ranges are also possible. For example, in some embodiments, the percent by mass of metal in the one or more material is greater than or equal to 0.1 wt % and less than or equal to 95 wt %.

The 3D printed scaffolds disclosed herein may, according to some embodiments, comprise a surface charge. The surface charge of the 3D printed scaffold may be negative, neutral, or positive. In some cases, the surface charge may be an intrinsic property of the material used to form the scaffold. For example, 3D printed scaffolds printed from a material with a neutral charge, such as quartz (e.g., SiO2), will have a neutral charge. Those skilled in the art will understand, however, that the surface charge of the 3D printed scaffold may be altered using known techniques in the art (e.g., altered from neutral to positive, neutral to negative, positive to negative, positive to neutral, negative to neutral, or negative to positive). For example, in some embodiments, exposure of the 3D printed scaffold to an acidic solution (e.g., loading a radionuclide (e.g., parent radionuclide) dissolved in an acid solution onto the scaffold) produces a positively charged surface. Without wishing to be bound by any particular theory, it is believed that this positive surface charge is due to the presence of hydrogen ions (H+) that preferentially coordinate with exposed atoms that are electronegative (e.g., have an available pair of lone electrons). In other words, H+ ions will associate with surfaces that contain electronegative atoms (e.g., nitrogen and oxygen), thus introducing positive charges to the surface of the scaffold.

Alternatively, or additionally, plasma treatment may be used to change the surface charge of a 3D printed scaffold. For example, in some embodiments, oxygen plasma treatment of quartz surfaces converts quartz surface siloxane groups into surface hydroxyl groups (—OH groups) and/or oxyanions (e.g., —O⁻ groups). These newly formed surface groups can be used (1) to adsorb various charged ligands (e.g., negatively charged ligands, positively charged ligands, or neutral ligands) onto the scaffold surface, and/or (2) to chemically conjugate various ligands to the scaffold surface (e.g., organofunctional silanes can be reacted with exposed surface hydroxyl groups, wherein the organofunctional domain comprises a negative, neutral, or positive charge). In some embodiments, plasma treatment may be used to alter the surface charge of any one of the 3D printed scaffolds (or their layers) disclosed herein (e.g., quartz scaffolds, zirconia scaffolds, stainless steel scaffolds, and Inconel scaffolds).

Layer-by-layer (LBL) deposition may also be used to alter the surface charge of any one of the 3D printed scaffolds, as disclosed herein. LBL deposition is an art recognized technique in which an exposed surface is repeatedly exposed to polyelectrolytes of alternating charge. As used herein, the term “polyelectrolyte” refers to any macromolecular material (e.g., a polymer, carbohydrates, polysaccharides, etc.) with repeated units that have the ability to dissociate in ionizing solvents (e.g., water). For example, a negative surface charge may be created by repeatedly exposing a 3D printed scaffold comprising a positive surface charge (e.g., zirconia) to a first solution comprising an anionic polyelectrolyte (e.g. polyacrylic acid, polyglutamic acid, sulfonated dextran, etc.,) followed by a second solution comprising a cationic polyelectrolyte (poly-L-lysine, polyethylenimine, PEI, gelatin, chitosan, cationic cellulose). One coating cycle corresponds deposition of one layer of each polyelectrolyte (e.g., one anionic polyelectrolyte layer deposited followed by one cationic polyelectrolyte layer deposited). Again, without wishing to be bound by any particular theory, it is generally believed that high coating cycles (e.g., >10) are useful for producing stable multilayer coatings over the target surface. Accordingly, in some embodiments, the 3D printed scaffold is exposed to greater than or equal to 1, greater than or equal to 5, greater than or equal to 10, greater than or equal to 15, greater than or equal to 20, or greater than or equal to 25, coating cycles. In some embodiments, the 3D printed scaffold is exposed to less than or equal to 25, less than or equal to 20, less than or equal to 15, less than or equal to 10, less than or equal to 5 coating cycles, or less than or equal to 1 coating cycles. Combinations of the above-referenced ranges are also possible. For example, in some embodiments, the 3D printed scaffold is exposed to greater than or equal to 1 and less than or equal to 25 coating cycles.

In some embodiments, the 3D printed substrates comprises one or more radionuclides. The terms “radionuclide”, “nuclide”, “radioactive isotope”, “radioisotope”, and “radioactive nuclide” are synonymous, as used herein, and refer to an isotope capable of undergoing radioactive decay for a certain period of time. Radioactive decay may comprise emission of gamma radiation, emission of a conversion electron, emission of Auger electrons, or emission of a new particle, such as an alpha particle or a beta particle, and it may occur over a period of fractions of a second to billions of years. In some embodiments, the radionuclide exhibits gamma emission. In some embodiments, the radionuclide exhibits beta emission. Beta decay may be beta negative decay (β⁻, i.e., an electron) or beta positive decay (β⁺, i.e., a positron). In other embodiments, the radionuclide exhibits alpha emission. In certain cases, the radionuclide is a multiple radiation emitter (e.g., gamma and alpha emission or alpha and beta emission). “Metal radionuclide” and the like refers to a radioisotope of a metal atom. Examples of metal radionuclides are provided herein.

The term “radioactive decay chain”, “radioactive cascade”, and “decay chain” are synonymous, as used herein, and refer to a series of radioactive decays of different radioactive decay products as a sequential series of transformations. The first radionuclide in each decay series is referred to as the parent radionuclide which undergoes decay to form a daughter isotope. Thus, the daughter radionuclide of a first decay becomes the parent radionuclide for the next decay in the sequence. The daughter isotope of a daughter isotope may be referred to as a granddaughter isotope. Alternatively, the daughter isotope may be an isotope that does not undergo further decay.

The term “alpha emitter” refers to a radionuclide that exhibits alpha emission, as described above. In some embodiments, the radionuclide is an alpha emitter selected from the group consisting of: ¹⁴⁹Tb, ²¹⁰Pb, ²¹¹At, ²¹²Bi, ²¹³Bi, ²¹²Pb, ²²³Ra, ²²⁴Ra, ²²⁵Ac, ²²⁶Th, ²²⁷Th, ²²⁸Th, and ²³⁰U.

The term “beta emitter” refers to a radionuclide that exhibits beta emission, as described above. In some embodiments, the radionuclide is a beta emitter selected from the group consisting of: ⁶⁸Ga, ⁸²Rb, ¹⁶⁶Ho, ⁸⁹Zr, ⁶¹Cu, ⁶⁴Cu, ⁹⁰Y, ¹⁵³Sm, ¹⁶¹Tb, ¹⁷⁷Lu, ¹⁸⁶Re, ¹⁸⁸Re, ¹⁶⁶Ho, ²³²Th, ²²⁸Ra, ²²⁸Ac, ²²⁸Th, ²²⁴Ra, ²²⁰Rn, ²¹²Pb, ²¹²Bi, ²¹²Po, and ²⁰⁸Ti.

The term “gamma emitter” refers to a radionuclide that exhibits gamma emission, as described above. In some embodiments, the radionuclide is a gamma emitter selected from the group consisting of ⁷Be, ²²Na, ²⁴Na, ⁵⁴Mn, ⁵⁷Co, ⁶⁰Co, ⁶⁶Ga, ⁶⁸Ga, ^{99m}Tc, ¹⁰³Pd, ¹¹¹In, ¹¹²Ag, ¹¹³Sn, ¹³²Te, ¹²⁵I, ¹³¹I, ¹³³Xe, ¹³⁴Cs ¹³⁷Cs, ¹³³Ba, ¹⁴⁴Ce, ²⁰¹Th, ²⁰³Pb, ²²²Rn, ²²⁶Ra, and ²⁴¹Am.

In some embodiments the radionuclide is selected from the group consisting of ¹⁴⁹Tb, ²¹⁰Pb, ²¹ At, ²¹²Bi, ²¹³Bi, ²¹²Pb, ²¹⁴Pb, ²²³Ra, ²²⁴Ra, ²²⁵Ac, ²²⁶Th, ²²⁷Th, ²²⁸Th, ²³⁰U, ⁶⁸Ga, ⁸²Rb, ¹⁶⁶Ho, ⁸⁹Zr, ⁶¹Cu, ⁶⁴Cu, ⁹⁰Y ¹⁵³Sm, ¹⁶¹Tb, ¹⁷⁷Lu, ¹⁸⁶Re, ¹⁸⁸Re, ¹⁶⁶Ho, ²³²Th, ²²⁸Ra, ²²⁸Ac, ²²⁸Th, ²²⁴Ra, ²²⁰Rn, ²¹²Pb ²¹²Bi, ²¹²Po, ²⁰⁸Ti, ⁷Be, ²²Na, ²⁴Na, ⁵⁴Mn, ⁵⁷Co, ⁶⁰Co, ⁶⁶Ga, ⁶⁸Ga, ^{99m}Tc, ¹⁰³Pd, ¹¹¹In, ¹¹²Ag, ¹¹³Sn, ¹³²Te, ¹²⁵I, ¹³¹I, ¹³³Xe, ¹³⁴Cs, ¹³⁷Cs, ¹³³Ba, ¹⁴⁴Ce, ²⁰¹Th, ²⁰³Pb, ²²²Rn, ²²⁶Ra, and ²⁴¹Am. In some embodiments, the radionuclide is ²²⁸Th. In some embodiments, the radionuclide is ²²⁵Ac.

In some embodiments, one or more radionuclides is any radionuclide that comprises a gaseous intermediate in its decay chain. In some embodiments, the gaseous intermediate is an isotope of radon. Those of skill in the art will understand that there are 39 known isotopes of radon (₈₆Rn) ranging from ¹⁹³Rn to ²³¹Rn, with ²²²Rn being the most stable (e.g., half life of about 3.8 days). In some embodiments, the gaseous intermediate is ²²²Rn. Those of skill in the art will know that ²²²Rn is an intermediate in the decay chain of ²³⁸U, ²³⁴Th, ^{234m}Pa ²³⁴U, ²³⁰Th, and ²²⁶Ra. Accordingly, in some embodiments, the radionuclide comprises ²³⁸U, ²³⁴Th, ^{234m}Pa ²³⁴U, ²³⁰Th, and ²²⁶Ra. In some embodiments, the gaseous intermediate is ²²⁰Rn. Again, the skilled artisan will understand that ²²⁰Rn is an intermediate in the decay chain of ²³²Th, ²²⁸Ra, ²²⁸Ac, ²²⁸Th, and ²²⁴Ra. Accordingly, in some embodiments, the radionuclide comprises ²³²Th, ²²⁸Ra, ²²⁸Ac, ²²⁸Th, and ²²⁴Ra.

In some embodiments, the one or more radionuclides comprises a half-life. As used herein, the term “half-life” refers to the amount of time it takes for one-half of a radionuclide to decay. Those of skill in the art will understand, and appreciate, that each radionuclide has a characteristic, constant half-life (t_{1/2}) that defines the time required for half of the atoms in a sample of said radionuclides to decay. Half-lives are commonly used in the art, for example, to determine how long a sample comprising a particular radionuclide will be available and/or how long a sample containing an undesirable or dangerous radionuclide must be stored before it decays to a low-enough radiation level that it is no longer a problem. In some embodiments, the half-life of ²²⁸Th is about 1.9 years. In some embodiments, the half-life of ²²⁴Ra is about 3.6 days. In some embodiments, the half-life of ²²⁰Rn is about 55 seconds. In some embodiments, the half-life of ²¹⁶Po is about 0.14 seconds. In some embodiments, the half-life of ²¹²Pb is about 10.6 hours.

As described more in detail below, in some embodiments, the one or more radionuclide is dissolved in a solution (e.g., an acidic solution) and subsequently applied to a 3D printed scaffold. Without wishing to be bound by any particular theory, it is believed that dissolution of the one or more radionuclides within said solution may produce a radionuclide salt. For example, dissolution of ²²⁸Th in a solution of nitric acid (e.g., HNO₃) produces thorium(IV) nitrate (²²⁸Th(NO₃)₄). Accordingly, in some embodiments, the one or more radionuclide comprises a radionuclide salt. In some embodiments, the radionuclide salt is ²²⁸Th(NO₃)₄. In some embodiments, the radionuclide salt is actinium nitrate (²³⁴Ac(NO₃)₃. Other radionuclide salts are also contemplated herein. In some embodiments, however, the one or more radionuclide is not a salt.

Other aspects of the disclosure relate to one or more methods of using a 3D printed scaffold. In some embodiments, the methods are directed toward capturing a parent radionuclide using any one of the 3D printed scaffolds disclosed herein. The skilled artisan will understand that the parent radionuclide may be any radionuclide, or combination of radionuclides, disclosed herein (e.g., ¹⁴⁹Tb, ²¹⁰Pb, ²¹ At, ²¹²Bi, ²¹³Bi, ²¹²Pb, ²¹⁴Pb, ²²³Ra, ²²⁴Ra, ²²⁵Ac, ²²⁶Th, ²²⁷Th, ²²⁸Th, ²³⁰U, ⁶⁸Ga, ⁸²Rb ¹⁶⁶Ho, ⁸⁹Zr, ⁶¹Cu, ⁶⁴Cu, ⁹⁰Y, ¹⁵³Sm ¹⁶¹Tb, ¹⁷⁷Lu, ¹⁸⁶Re, ¹⁸⁸Re, ¹⁶⁶Ho, ²³²Th, ²²⁸Ra, ²²⁸Ac, ²²⁸Th, ²²⁴Ra, ²²⁰Rn, ²¹²Pb, ²¹²Bi, ²¹²Po, ²⁰⁸Ti, ⁷Be, ²²Na, ²⁴Na, ⁵⁴Mn, ⁵⁷Co, ⁶⁰Co, ⁶⁶Ga, ⁶⁸Ga, ^{99m}Tc, ¹⁰³Pd, ¹¹¹In, ¹¹²Ag, ¹¹³Sn, ¹³²Te, ¹²⁵I, ¹³¹I, ¹³³Xe, ¹³⁴Cs, ¹³⁷Cs, ¹³³Ba, ¹⁴⁴Ce, ²⁰¹Th, ²⁰³Pb, ²²²Rn, ²²⁶Ra, and ²⁴¹Am). In some embodiments, the parent radionuclide is ²²⁸Th. In some embodiments, the parent radionuclide is ²²⁴Ra.

In some embodiments, the methods comprise incubating the 3D printed scaffold in a solution. In some embodiments, the solution is used to alter the surface charge of the 3D printed scaffold (e.g., from neutral to positive). For example, as described in detail above, it is believed that incubation in acidic solutions (e.g., HNO₃) converts neutrally charged surfaces to positively charged surfaces. Alternatively, it is believed that repeatedly incubating the 3D printed scaffolds in alternating solutions of cationic polyelectrolytes and anionic polyelectrolytes creates an electrostatically stabilized multilayer coating that facilitates conversion of a negatively charged surface to a positively charged surface, and vice versa.

In some embodiments, the solution is an acidic solution. Exemplary acidic solutions include, but are not limited to, hydrochloric acid (HCl), sulfuric acid (H₂SO₄), phosphoric acid (H₃PO₄), and nitric acid (HNO₃). In some embodiments, the concentration of the acidic solution is between 0.1 M and 5 M. In some embodiments, the concentration of the acidic solution is greater than or equal to 0.1 M, greater than or equal to 0.25 M, greater than or equal to 0.5 M, greater than or equal to 1 M, greater than or equal to 2 M, greater than or equal to 3 M, greater than or equal 4 M, or greater than or equal to 5 M. In some embodiments, the concentration of the acidic solution is less than or equal to 5 M, less than or equal to 4 M, less than or equal to 3 M, less than or equal to 2 M, less than or equal to 1 M, less than or equal to 0.5 M, or less than or equal to 0.25 M, less than or equal 0.1 M. Combinations of the above-referenced ranges are also possible. For example, in some embodiments, the concentration of the acidic solution is greater than or equal to 0.1 M and less than or equal to 5 M. Other ranges are also possible.

In some embodiments, the solution is used to disperse a radionuclide (e.g., parent radionuclide). Accordingly, in some embodiments, the solution comprises the radionuclide (e.g., parent radionuclide). In some embodiments, the radionuclide (e.g., parent radionuclide) in the solution comprises ²²⁰Rn in its decay chain. In some embodiments, the radionuclide (e.g., parent radionuclide) is ²²⁴Ac. In some embodiments, the radionuclide (e.g., parent radionuclide) in the solution comprises ²²²Rn in its decay chain. In some embodiments, the radionuclide (e.g., parent radionuclide) is ²²⁸Th or ²²⁴Ra.

The skilled artisan will understand that incubating a 3D printed scaffold in a solution comprising a radionuclide (e.g., parent radionuclide) may be used to facilitate attachment of the radionuclide (e.g., parent radionuclide) onto one or more exposed surfaces of the 3D printed scaffold (e.g., to produce a radionuclide-loaded 3D printed scaffold). Accordingly, in some embodiments, the methods comprise attaching the radionuclide (e.g., parent radionuclide) onto one or more of the exposed surfaces of the 3D printed scaffold to produce a radionuclide-loaded 3D printed scaffold. For example, as described in more detail above, incubating the 3D printed scaffold in a solution comprising the radionuclide (e.g., parent radionuclide) facilitates attachment of the radionuclide (e.g., parent radionuclide) to one or more exposed surfaces of the 3D printed scaffold via adsorption (e.g., physisorption, chemisorption, electrostatic, etc.).

Other methods may also be used to facilitate attachment of the radionuclide (e.g., parent radionuclide) to one or more exposed surfaces of the 3D printed scaffold. For example, in some embodiments, the radionuclide (e.g., parent radionuclide) is electrodeposited from the solution comprising the radionuclide (e.g., parent radionuclide) onto one or more exposed surfaces of the 3D printed scaffolds. Without wishing to be bound by any particular theory, electrodeposition (e.g., electroplating) is a process for producing a metal coating on a solid substrate through the reduction of cations of that metal by means of a direct electric current (e.g., electrolytic cell). In this scenario, the part to be coated (e.g., 3D printed scaffold comprising zirconia) acts as the cathode (e.g., negative electrode) of an electrolytic cell, the solution comprising the radionuclide (e.g., parent radionuclide) acts as the electrolyte and comprises a salt (e.g., ²²⁸Th(NO₃)₄) of the radionuclide to be coated (e.g., ²²⁸Th), and any inert conductive material acts as the anode of the cell. Upon application of a current, the metal cations in solution (²²⁸Th⁴⁺) become reduced at the cathode to the zero valence state. For example, the electrolyte for the deposition of ²²⁸Th is thorium(IV)nitrate (²²⁸Th(NO₃)₄), which dissociates into (²²⁸Th⁴⁺) and NO₃⁻. At the cathode (e.g., the one or more exposed surfaces of the 3D printed scaffold) the ²²⁸Th⁴⁺ is reduced to metallic ²²⁸Th by gaining two electrons. In some embodiments, the attachment of a radionuclide (e.g., parent radionuclide) to one or more exposed surfaces of the 3D printed scaffold produces a radionuclide-loaded 3D printed scaffold.

In some embodiments, one or more methods disclosed herein are directed toward using radionuclide-loaded 3D printed scaffolds in combination with a radionuclide generator to collect a gas-phase intermediate daughter radionuclide at a location that is different than the 3D printed scaffold. Any suitable generator capable of using the radionuclide-loaded 3D printed scaffolds, as disclosed herein, to capture a gas-phase intermediate daughter radionuclide at a location that is different than the 3D printed scaffold may be used as disclosed herein, such as those disclosed in U.S. Patent Application No. 63/458,296, entitled “Radionuclide Generator” filed on Apr. 10, 2023.

In some embodiments, the methods involve placing a radionuclide-loaded 3D printed scaffold into a radionuclide generator, wherein the radionuclide comprises ²²⁰Rn in its decay chain. As disclosed elsewhere herein, the skilled artisan will understand that ²²⁰Rn is an intermediate in the decay chain of ²³²Th, ²²⁸Ra, ²²⁸Ac, ²²⁸Th, and ²²⁴Ra. Accordingly, in some embodiments, the radionuclide-loaded 3D printed scaffold comprises ²³²Th, ²²⁸Ra, ²²⁸Ac, ²²⁸Th, and ²²⁴Ra. In some embodiments, the methods involve allowing ²³²Th, ²²⁸Ra, ²²⁸Ac, ²²⁸Th, and ²²⁴Ra to decay to ²²⁰Rn and allowing the ²²⁰Rn to diffuse to a location different than the 3D printed scaffold (e.g., FIG. 9A-9B). In some embodiments, the ²²⁰Rn diffuses to a location different than the radionuclide-loaded 3D scaffold at an accelerated rate. Any method to accelerate the diffusion of ²²⁰Rn to a location different than the radionuclide-loaded 3D scaffold known by the skilled artisan may be used herein. Non-limiting examples include increasing the operating temperature of the radionuclide generator, using a mass accelerator (e.g., an electrostatic accelerator) or using an inert gas stream (e.g., argon, nitrogen, helium, etc.) to carry the ²²⁰Rn to a location different than the 3D printed scaffold.

In some embodiments, the methods involve collecting the ²²⁰Rn at the location different than the location of the scaffold. Any known, or yet to be determined, method for collecting the ²²⁰Rn at a location different than the radionuclide-loaded scaffold may be used herein. An exemplary embodiment is shown in FIG. 9B in which exemplary radionuclide generator 920 comprises source module 925 comprising radionuclide-loaded 3D printed scaffold 930 placed within collection vessel 935. In some embodiments, the ²²⁰Rn collected within the collection vessel further decays to one or more products, for example, ²¹²Pb. In some embodiments, the collection vessel comprises ²¹²Pb. In some embodiments, the collection vessel comprises a mixture of ²²⁰Rn and ²¹⁶Po. In some embodiments, the collection vessel comprises a mixture ²²⁰Rn, ²¹⁶Po, and ²¹²Pb. In some embodiments, the collection vessel comprises a mixture of ²²⁰Rn, ²¹⁶Po, ²¹²Pb, and ²¹²Bi. It should be noted, however, that because the collection vessel only contains ²²⁰Rn and its daughter radionuclides, that the collection vessel does not contain the radionuclide (e.g., parent radionuclide) loaded within the 3D printed scaffold.

The amount of time needed for one or more parent radionuclides to decay to a daughter nucleotide (e.g., ²²⁰Rn), and for the daughter nucleotide to subsequently diffuse to a location (e.g., a second location) different than that from the location of a radionuclide-loaded 3D printed scaffold (e.g., a first location) will depend on the chemical properties of the radionuclide (e.g., parent radionuclide) and the design of the respective radionuclide generator. In some embodiments, the radionuclide (e.g., parent radionuclide) is allowed to decay (e.g., while in the generator; e.g., before a daughter nucleotide is collected at the second location) for greater than or equal to 1 hour, greater than or equal to 2 hours, greater than or equal to 3 hours, greater than or equal to 4 hours, greater than or equal to 5 hours, greater than or equal to 6 hours, greater than or equal to 7 hours, greater than or equal to 8 hours, greater than or equal to 9 hours, greater than or equal to 10 hours, greater than or equal to 12 hours, greater than or equal to 24 hours, greater than or equal to 36 hours, or greater than or equal to 48 hours. In some embodiments, the radionuclide (e.g., parent radionuclide) is allowed to decay for less than or equal to 48 hours, less than or equal to 36 hours, less than or equal to 24 hours, less than or equal to 12 hours, less than or equal to 10 hours, less than or equal to 9 hours, less than or equal to 8 hours, less than or equal to 7 hours, less than or equal to 6 hours, less than or equal to 5 hours, less than or equal to 4 hours, less than or equal to 3 hours, less than or equal to 2 hours, or less than or equal to 1 hour. Combinations of the above-referenced ranges are also possible. For example, in some embodiments, the radionuclide (e.g., parent radionuclide) is allowed to decay for greater than or equal to 1 hour and less than or equal to 48 hours.

In some embodiments, the daughter radionuclide (e.g., ²²⁰Rn) is allowed to diffuse to a location different than the radionuclide-loaded 3D scaffold for greater than or equal to 1 hour, greater than or equal to 2 hours, greater than or equal to 3 hours, greater than or equal to 4 hours, greater than or equal to 5 hours, greater than or equal to 6 hours, greater than or equal to 7 hours, greater than or equal to 8 hours, greater than or equal to 9 hours, greater than or equal to 10 hours, greater than or equal to 12 hours, greater than or equal to 24 hours, greater than or equal to 36 hours, or greater than or equal to 48 hours. In some embodiments, the radionuclide (e.g., parent radionuclide) is allowed to diffuse to a location different than the radionuclide-loaded 3D scaffold for less than or equal to 48 hours, less than or equal to 36 hours, less than or equal to 24 hours, less than or equal to 12 hours, less than or equal to 10 hours, less than or equal to 9 hours, less than or equal to 8 hours, less than or equal to 7 hours, less than or equal to 6 hours, less than or equal to 5 hours, less than or equal to 4 hours, less than or equal to 3 hours, less than or equal to 2 hours, or less than or equal to 1 hour. Combinations of the above-referenced ranges are also possible. For example, in some embodiments, the radionuclide (e.g., parent radionuclide) is allowed to diffuse to a location different than the radionuclide-loaded 3D scaffold for greater than or equal to 1 hour and less than or equal to 48 hours.

Other aspects of the disclosure relate to printing a 3D printed scaffold using continuous or semi-continuous additive manufacturing (e.g., 3D printing) techniques. Additive manufacturing techniques provide for the layer-by-layer formation of an object. As each layer is formed, precise details of the object are fabricated using a bottom-up approach to form details in the object, one layer at a time. Because additive manufacturing techniques build an object layer by layer, multiple iterations may be implemented in order to form the full object. The skilled artisan will understand and appreciate that any suitable additive manufacturing technique known in the art may be used to print the scaffolds disclosed herein. For example, zirconia-based scaffolds can be printed using methods such as selective laser sintering (SLS), selective laser melting (SLM), stereolithography (SLA), ink-jet printing, and digital light processing (DLP).

In some embodiments, the scaffold is printed using SLS. Generally, SLS works by dispensing a powder in a thin layer on top of a platform inside of a build chamber. The printer preheats the powder to a temperature somewhat below the melting point of the raw material, which makes it easier for the laser to raise the temperature of specific regions of the powder bed as it traces the model to solidify a part. The laser scans a cross-section of the 3D model, heating the powder to just below or right at the melting point of the material. This fuses the particles together mechanically to create one solid part. The unfused powder supports the part during printing and eliminates the need for dedicated support structures. The platform then lowers by one layer into the build chamber, typically between 50 to 200 microns, and the process repeats for each layer until parts are complete.

In some embodiments, the scaffold is printed using SLM. SLM is similar to SLS. During the printing process, a laser beam melts and fuses various metallic powders together. As the laser beam hits a thin layer of the material, it selectively joins or welds the particles together. After one complete print cycle, the printer adds a new layer of powered material to the previous one. The object is then lowered by the precise amount of the thickness of a single layer and the process repeated until the object is printed.

In some embodiments, the scaffold is printed using SLA. Stereolithography is an additive manufacturing process that works by focusing an ultraviolet (UV) laser on to a vat of photopolymer resin. With the help of computer aided manufacturing or computer-aided design (CAM/CAD) software, the UV laser is used to draw a pre-programmed design or shape on to the surface of the photopolymer vat. Photopolymers are sensitive to ultraviolet light, so the resin is photochemically solidified and forms a single layer of the desired 3D object. Then, the build platform lowers one layer and a blade recoats the top of the tank with resin. This process is repeated for each layer of the design until the 3D object is complete.

In some embodiments, the scaffold is printed using ink-jet printing. Ink-jet printing works by extruding a printing material through a small nozzle within a print head. As the print head raster scans over a surface, multiple layers can be build in a layer-by-layer process until the desired object is created. One advantage of ink-jet printing is that the print heads are configured to print a variety of liquid materials (e.g., polymers) or solid suspension (e.g., nanoparticles). In this way, polymers, dielectric nanoparticles, and conductive nanoparticles may be printed using this technique. DLP works by lowering a build platform into a transparent resin tank filled with liquid photopolymer. A high-resolution projector then shines a UV light onto the build platform in the same shape as the cross-section of the part layer. The cross-section projection is created with an array of microscopic mirrors called a DMD that direct light only where needed. The only photopolymer that gets cured is that which is both illuminated and physically in contact with a solid surface (the build platform or a previous layer). Once a layer is complete, the build platform moves up (or down) by the thickness of one layer, and the process is repeated until the part is completed.

Similarly, quartz-based scaffolds can be printed using lithography-based ceramic manufacturing (LSM) as described by Scheithauer, U., Schwarzer, E., Moritz, T. et al. Additive Manufacturing of Ceramic Heat Exchanger: Opportunities and Limits of the Lithography-Based Ceramic Manufacturing (LCM). J. of Materi Eng and Perform 27, 14-20 (2018), which is hereby incorporated by reference in its entirety.

In some embodiments, the scaffold is printed using LCM. LCM works by dispensing a ceramic-loaded liquid (slurry) into a transparent vat. A movable build platform is dipped into the slurry from above, which is then selectively exposed to visible blue light from below the tub. The layered image is produced via a digital micromirror device (DMD) in combination with an image projection system. By repeating this process, a three-dimensional green part can be created layer by layer. After thermal post-processing, the binder is removed and the components are sintered, resulting in fully dense ceramic components. LCM technology utilizes decades of experience in processing ceramic powders. By using the same powders and ovens as injection molding, ceramic components with excellent mechanical properties and surface qualities can be produced.

### EXAMPLES

Example 1 describes exemplary 3D printed scaffolds comprising a first layer, a second layer, and a radionuclide, as contemplated herein, according to some embodiments (FIGS. 1A and 1B).

Example 2 describes a first layer of a 3D printed scaffold, wherein the first layer comprises a plurality of substantially parallel first structures aligned along a first axis, as contemplated herein, according to some embodiments (FIG. 2).

Example 3 describes a second layer of a 3D printed scaffold, wherein the second layer comprises a plurality of substantially parallel second structures aligned along a second axis, as contemplated herein, according to some embodiments (FIG. 3).

Example 4 describes a 3D printed scaffold comprising a first layer comprising a plurality of substantially parallel first structures aligned along a first axis and a second layer comprising the plurality of substantially parallel second structures aligned along a second axis (FIG. 4A). Example 4 also illustrates a first angle formed between the first axis and the second axis, according to some embodiments.

Example 5 describes a 3D printed scaffold comprising a first layer comprising a plurality of substantially parallel first structures aligned along a first axis and a second layer comprising the plurality of substantially parallel second structures aligned along a second axis (FIG. 4B). Example 5 also illustrates a second angle formed between the first axis and the second axis, according to some embodiments.

Example 6 describes a 3D printed scaffold comprising a first layer, a second layer, and a radionuclide (FIG. 4C) as contemplated herein. Example 6 also illustrates a cross-sectional view of said 3D printed scaffold further illustrating the deposition of the radionuclide on one or more exposed surfaces of the first and/or second layers (FIG. 4D), according to some embodiments.

Example 7 describes the observed pore shapes contemplated to formed in the spacings between the first and second substantially parallel structures in the first and second layers aligned along a first axis and second axis, respectively, when viewed from the top position for a 3D printed scaffolds. In some embodiments, the angle between the first axis and second axis is between 0 and 90 degrees. Contemplated pore shapes include, but are not limited to, rectangle pore (FIG. 5A), diamond pore (FIG. 5B), and square (FIG. 5C), according to some embodiments.

Example 8 describes exemplary nonporous top and/or bottom layer of a 3D printed scaffold, as contemplated herein. In some embodiments, the nonporous layer may comprises a plurality of grooves and/or channels, according to some embodiments.

Example 9 describes a 3D printed scaffold, as contemplated herein, comprising a closed top and bottom geometry. In some embodiments, a closed top and bottom geometry comprises a first layer and/or a second layer positioned within a nonporous bottom layer, a nonporous top layer, and a nonporous side layer (FIG. 7A-7C), according to some embodiments.

Example 10 describes a 3D printed scaffold, as contemplated herein, comprising an open top geometry. In some embodiments, an open top geometry comprises a first layer and/or a second layer positioned with a nonporous bottom layer and a nonporous side layer (FIG. 8A-8B), according to some embodiments.

Example 11 describes an exemplary radionuclide generator that may be used in combination with any of the 3D printed scaffolds (FIG. 9A-9C), as contemplated herein, according to some embodiments.

### Equivalents and Scope

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A 3D printed scaffold for attaching a radionuclide, comprising:
a first layer comprising a plurality of substantially parallel first structures, wherein the plurality of first structures are substantially aligned along a first axis, and wherein an average spacing between adjacent first structures is at least 0.1 mm and less than or equal to 0.5 mm;
a second layer comprising a plurality of substantially parallel second structures, wherein the plurality of second structures are substantially aligned along a second axis, and wherein an average spacing between adjacent second structures is at least 0.1 mm and less than or equal to 0.5 mm; and
a radionuclide attached to one or more exposed surfaces of the first structures and/or one or more exposed surfaces of the second structures,
wherein an angle between the first axis and second axis is greater than 0 degrees and less than or equal to 90 degrees,
wherein the plurality of first structures has an average cross-sectional area of at least 20 mm² and less than or equal to 2000 mm², and an average width of at least 5 mm and less than or equal to 50 mm,
wherein a ratio between the average width of the first structures and the average spacing between adjacent first structures is at least 0.1 mm and less than or equal to 0.5 mm, and
wherein the 3D printed scaffold comprises an infill density of at least 10% and less than or equal to 90%.

2. A method for capturing a radionuclide using a 3D printed scaffold, the method comprising:
with a 3D printed scaffold, performing the steps of:
incubating the 3D printed scaffold in a solution comprising the radionuclide that comprises ²²⁰Rn in its decay chain,
attaching the radionuclide onto one or more exposed surfaces of the 3D printed scaffold to produce a radionuclide-loaded 3D printed scaffold,
wherein the 3D printed scaffold comprises:
(i) a first layer comprising a plurality of substantially parallel first structures, wherein the plurality of first structures are substantially aligned along a first axis, and wherein an average spacing between adjacent first structures is at least 0.1 mm and less than or equal to 0.5 mm;
(ii) a second layer comprising a plurality of substantially parallel second structures, wherein the plurality of second structures are substantially aligned along a second axis, and wherein an average spacing between adjacent second structures is at least 0.1 mm and less than or equal to 0.5 mm; and
wherein an angle between the first axis and second axis is greater than 0 degrees and less than or equal to 90 degrees,
wherein the plurality of first structures has an average cross-sectional area of at least 20 mm² and less than or equal to 2000 mm², and an average width of at least 5 mm and less than or equal to 50 mm,
wherein a ratio between the average width of the first structures and the average spacing between adjacent first structures is at least 0.1 mm and less than or equal to 0.5 mm, and
wherein the 3D printed scaffold comprises an infill density of at least 10% and less than or equal to 90%.

3. A method, comprising
(i) placing a radionuclide-loaded 3D printed scaffold into a radionuclide generator,
wherein the radionuclide-loaded 3D printed scaffold comprises:
(a) a first layer comprising a plurality of substantially parallel first structures, wherein the plurality of first structures are substantially aligned along a first axis, and wherein an average spacing between adjacent first structures is at least 0.1 mm and less than or equal to 0.5 mm;
(b) a second layer comprising a plurality of substantially parallel second structures, wherein the plurality of second structures are substantially aligned along a second axis, and wherein an average spacing between adjacent second structures is at least 0.1 mm and less than or equal to 0.5 mm; and
(c) a radionuclide that comprises ²²⁰Rn in its decay chain;
(ii) allowing the radionuclide to decay to gaseous ²²⁰Rn;
(iii) allowing the gaseous ²²⁰Rn to diffuse to a location different than the 3D printed scaffold; and
(iv) collecting the ²²⁰Rn at the location different than the scaffold.

4. The scaffold of claim 1, further comprising one or more nonporous layers.

5. The scaffold of claim 4, wherein the one or more nonporous layers is a nonporous bottom layer.

6. The scaffold of claim 4, wherein the one or more nonporous layers is a nonporous top layer.

7. The scaffold of claim 4, wherein the one or more nonporous layers is a nonporous side layer.

8. The scaffold of claim 4, wherein the one or more nonporous layers comprises grooves and/or channels.

9. The scaffold of claim 1, wherein the angle between the first axis and the second axis is 90 degrees such that the plurality of first structures and the plurality of second structures form a plurality of rectangular pores.

10. The scaffold of claim 9, wherein the rectangular pore has an area of at least 0.1 mm and less than or equal to 1 mm.

11. The scaffold of claim 9, wherein the rectangular pore has an aspect ratio of at least 1:1 and less than or equal to 10:1.

12. The scaffold of claim 9, wherein the rectangular pore is a square pore.

13. The scaffold of claim 1, wherein the 3D printed scaffold comprises at least four layers.

14. The scaffold of claim 1, wherein the layers of the 3D printed scaffold are positioned within a nonporous bottom layer, a nonporous top layer, and a nonporous side layer.

15. The scaffold of claim 1, wherein the one or more exposed surfaces comprise any surface of the plurality of first structures and/or the plurality of second structures.

16. The scaffold of claim 1, wherein the radionuclide is attached to the exposed surfaces of both the plurality of first structures and the plurality of the second structures.

17. The scaffold of claim 1, wherein the radionuclide is attached to one or more exposed surfaces of a nonporous bottom layer, a porous top layer, or a nonporous side layer.

18. The scaffold of claim 1, wherein the radionuclide is an alpha, beta or gamma emitter.

19. The scaffold of claim 1, wherein the radionuclide is selected from the group consisting of ¹⁴⁹Tb, ²¹⁰Pb, ²¹¹At, ²¹²Bi, ²¹³Bi, ²¹²Pb ²²³Ra, ²²⁴Ra ²²⁵Ac, ²²⁶Th, ²²⁷Th, ²²⁸Th, ²³⁰U, ⁶⁸Ga, ⁸²Rb, ¹⁶⁶Ho, ⁸⁹Zr, ⁶¹Cu, ⁶⁴Cu, ⁹⁰Y, ¹⁵³Sm, ¹⁶¹Tb ¹⁷⁷Lu, ¹⁸⁶Re, ¹⁸⁸Re, ¹⁶⁶Ho, ²³²Th, ²²⁸Ra, ²²⁸Ac, ²²⁸Th, ²²⁴Ra, ²²⁰Rn, ²¹²Pb, ²¹²Bi, ²¹²Po, ²⁰⁸Ti, ⁷Be, ²²Na, ²⁴Na, ⁵⁴Mn, ⁵⁷CO, ⁶⁰CO, ⁶⁶Ga, ⁶⁸Ga, ^{99m}Tc, ¹⁰³Pd, ¹¹¹In, ¹¹²Ag, ¹¹³Sn, ¹³²Te, ¹²⁵I, ¹³¹I, ¹³³Xe, ¹³⁴Cs, ¹³⁷Cs, ¹³³Ba, ¹⁴⁴Ce, ²⁰¹Th, ²⁰³Pb, ²²²Rn, ²²⁶Ra, and ²⁴¹Am.

20. The scaffold of claim 1, wherein the radionuclide is ²²⁸Th or ²²⁴Ra.

21. The scaffold of claim 1, wherein the radionuclide decays to produce a gaseous intermediate radionuclide.

22. The scaffold of claim 21, wherein the gaseous intermediate radionuclide is ²²⁰Rn.

23. The scaffold of claim 22, wherein the ²²⁰Rn diffuses to a location different than a location of the 3D printed scaffold.

24. The scaffold of claim 22, wherein the ²²⁰Rn further decays to a ²¹²Pb.

25. The scaffold of claim 24, wherein the ²¹²Pb is deposited onto a substrate different than the 3D printed scaffold.

26. The scaffold of claim 24, wherein the ²¹²Pb is deposited onto the 3D printed scaffold.

27. The scaffold of claim 26, wherein the ²¹²Pb covers greater than or equal to 1% or less than or equal to 10% of the one or more exposed surfaces of the 3D printed scaffold.

28. The scaffold of claim 1, wherein the 3D printed scaffold comprises zirconia.

29. The scaffold of claim 28, wherein the zirconia is yttria-stabilized zirconia.

30. The scaffold of claim 1, wherein the 3D printed scaffold comprises quartz (SiO₂).

31. The scaffold of claim 1, wherein the 3D printed scaffold comprise a positive surface charge.

32. The scaffold of claim 1, wherein the 3D printed scaffold comprises a negative surface charge.
